(19) United States
(12) Patent Application Publication
(10) Pub. No.: US 2026/0350796 A1
(43) Pub. Date: Nov. 5, 2026

(54) FOOT SUPPORT BASED ON MOBILE SCAN

(71) Applicant: Superfeet Worldwide LLC, Ferndale, WA (US)

(72) Inventors: Achal Aggarwal, Ferndale, WA (US); Sumit Raj, Ferndale, WA (US); Heather Allerdice-Gerow, Ferndale, WA (US); Michael Sullivan, Ferndale, WA (US)

(21) Appl. No.: 19/661,339

(22) Filed: Apr. 28, 2026

Related U.S. Application Data
(60) Provisional application No. 63/797,936, filed on Apr. 30, 2025.
(60) Provisional application No. 63/976,484, filed on Feb. 5, 2026.

(51) Int. Cl. G06T 7/00; H04N 23/60; H04N 23/63

(52) U.S. Cl. CPC G06T 7/0012; H04N 23/633; H04N 23/64; G06T 2207/10028; G06T 2207/30004; G06T 2207/30196

(57) ABSTRACT

Embodiments are directed toward selecting or generating a foot-support orthotic recommended for a foot. An image with depth information for the foot is obtained. The image with depth information is captured by a mobile smartphone or a tablet. Based on the image with depth information, characteristics of the foot are evaluated. A foot-support orthotic recommended for such characteristics is selected or generated. Accordingly, the evaluation and recommendation reduces complexity compared to processes that include a non-foot object of a known size in the image as a scaler.

### PRIORITY CLAIM

This application claims benefit of U.S. Provisional Application No. 63/797,936 entitled FOOT SUPPORT BASED ON MOBILE SCAN filed Apr. 30, 2025, and U.S. Provisional Application No. 63/976,484 entitled MOBILE FOOT SCAN FOR INSOLE CREATION filed Feb. 5, 2026. The entire contents of which are incorporated herein by reference in their entireties.

### FIELD OF THE INVENTION

The invention relates generally to evaluating foot characteristics and, more particularly, to evaluating foot characteristics based on a digital scan or digital photograph.

### BACKGROUND OF THE INVENTION

Many people, whether for athletics, standing, or walking, benefit from orthotics. Orthotics can hold or change the shape of a person's foot at rest or under load. Accordingly, it is important that the orthotic be sized and shaped to address the particular conditions of the person's feet, such as flat feet, high arches, or others. It is typically beneficial to scan the person's foot to reduce the subjectivity in evaluating the conditions of the foot, thereby increasing the likelihood of accurate measurements and a correct diagnosis.

Such scans typically require custom hardware that is specifically designed for scanning feet to enable such measurements and diagnoses. Some such hardware scans the foot from underneath, such as those taught in International Publication No. WO 2019/051329 or U.S. Pat. No. 9,460,557. Other such hardware scans the foot from multiple angles around the foot, such as those taught in European Patent No. EP 3,523,601 or U.S. Pat. No. 7,738,145. Ordinary consumers in need of such insoles or orthotics, however, do not possess such hardware, which is typically owned and operated by manufacturers, specialty fitters, or retailers, rather than individuals.

To increase access to measuring foot characteristics, some have attempted to make such measurements and diagnoses based on photographs of feet. Some such approaches require the user to take multiple photographs from different angles to obtain three-dimensional information from a stitched model based on a combination of the photographs, such as the approaches taught in U.S. Pre-Grant Publication No. 2021/0166411 or U.S. Pre-Grant Publication No. 2020/0275861. Others require the user to place a reference object in the photographs to enable identifying the scale of the image, such as the approaches taught in U.S. Pat. Nos. 11,816,806, 11,615,616, 10,564,628, or U.S. Pre-Grant Publication No. 2020/0275861. The higher the quantity or complexity of the steps that are required from the individual, however, the lower the likelihood that the individual correctly completes the process. These approaches therefore decrease the likelihood that the individual obtains the orthotics that the individual needs.

For these reasons, there is a need for evaluating characteristics of an individual's foot based on a digital scan (or digital photograph) taken with a mobile device by the individual. There is also a need for such evaluation to have a reduced quantity of steps taken by the individual. There is a further need for such evaluation to have a reduced complexity of steps taken by the individual.

### SUMMARY OF THE INVENTION

It is therefore an object of the present invention to provide a customized recommendation for a foot-support insole or orthotic based on an image of an individual's foot while reducing a quantity of steps taken by the individual. Footwear recommendations can also be made.

It is also an object of the present invention to achieve the above object and that also reduces the complexity of the steps taken by the individual.

It is another object of the present invention to achieve the above objects and that also enable providing the customized recommendation based on the image provided by a mobile smartphone or tablet, rather than customized hardware.

It is a further object of the present invention to achieve the above objects and that also provide dynamic instructions to the individual based on how the user arranged the mobile smartphone or tablet.

The invention achieves the above objects, as well as other objects and advantages that will become apparent from the description that follows, by selecting or generating a foot-support orthotic recommended based on evaluation of an image with depth information for a foot. The image with depth information is preferably captured by a mobile smartphone or a tablet or a camera configured to facilitate authorizing access to a device based on biometrics captured by the camera. Based on the image with depth information, one or more characteristics of the foot are evaluated. Based on the evaluation, a foot-support orthotic recommended for the foot is selected or generated. Accordingly, the invention facilitates customized selection or generation of the foot-support orthotic with reduced complexity compared to including a non-foot object of a known size in the image as a scaler. The invention also therefore enables providing the customized recommendation while determining the one or more foot characteristics based on the image provided by the mobile smartphone or tablet, rather than customized hardware.

In some embodiments, the depth information includes a depth map provided by the mobile smartphone or the tablet or camera. In some embodiments, the camera includes a TrueDepth camera system or similar depth camera.

In some embodiments, the image with depth information includes only a single still frame photograph with the depth information. Evaluating the foot characteristics is preferably based on only the single still frame photograph with the depth information. In some embodiments, the image lacks, as the scalar, non-foot objects of size known to the one or more computers evaluating the characteristics of the foot, such as a piece of paper or other prescribed object.

In some embodiments, real-time image data from the mobile smartphone, tablet, or camera is evaluated to determine whether parameters are satisfied, and the image is preferably obtained automatically responsive to the parameters being satisfied. The parameters preferably include (i) that the foot is present in a field of view and (ii) that a medial side of the foot faces the camera, mobile smartphone, or tablet.

In some embodiments, the method is performed in response to the mobile smartphone or the tablet accessing a website, rather than downloading an application other than a web browser.

In some embodiments, the mobile smartphone or the tablet displays a reference indicator showing where the foot should be positioned. The reference indicator is preferably superimposed on a real-time display of a field of view of a camera of the mobile smartphone or the tablet. The position of the reference indicator preferably dynamically varies based on an orientation of the mobile smartphone or the tablet, including dynamic adjustment of a height of the reference indicator based on an angle of the mobile smartphone or the tablet.

### BRIEF DESCRIPTION OF THE DRAWINGS

The preferred embodiment of the present invention is described in detail below with reference to the following drawings.

FIG. 1 shows a schematic representation of a foot-support selection or generation system according to principles of the invention.

FIG. 2 illustrates an overview flowchart of a process for selecting or generating foot supports, executed by the system of FIG. 1.

FIG. 3 shows a flow diagram of a process for obtaining a scan of a user's foot, executed by a photograph engine in the system of FIG. 1.

FIG. 4 illustrates a schematic representation of an environment having a mobile device through which the photograph can be obtained with the process of FIG. 3.

FIG. 5 shows a schematic elevational side view of the mobile device of FIG. 4.

FIG. 6 illustrates a flowchart of a process for obtaining the photograph of the user's foot, executed by the photograph engine in the system of FIG. 1.

FIG. 7 illustrates a schematic representation of a prompt that the system of FIG. 1 displays on the mobile device to encourage the user to enable photographing the user's foot.

FIG. 8 shows a schematic representation of training a model for identifying the user's foot, executed by a foot-identification engine of the system of FIG. 1.

FIG. 9 shows a schematic representation of a user interface that the system of FIG. 1 displays on the mobile device to notify the user that the system is obtaining the photograph of the user's foot.

FIG. 10 illustrates a flow diagram of a process for evaluating characteristics of the user's foot, executed by a foot-evaluation engine of the system of FIG. 1.

FIG. 11 shows a schematic representation of a scan of the users photographed foot by a filter engine of FIG. 1 to enable the foot measurements.

FIG. 12 shows a schematic representation of milestones in a process of a foot-evaluation engine of the system of FIG. 1 for obtaining the foot measurements.

FIG. 13 illustrates a schematic representation of measurements by the foot-evaluation engine of the system of FIG. 1 to facilitate selecting or generating a foot-support orthotic.

FIG. 14 shows a schematic representation of a system environment in which the system of FIG. 1 is implemented according to some embodiments.

FIG. 15 illustrates a schematic representation of a client computer of the system of FIG. 1 according to some embodiments.

FIG. 16 shows a schematic representation of a network computer of the system of FIG. 1 according to some embodiments.

FIGS. 17-21 illustrate schematic representations of screen captures of a mobile device of a user of the system of FIG. 1.

FIG. 22 shows a flow diagram for remotely controlling a scan of the user's foot according to the system of FIG. 1 and the process of FIG. 2.

FIGS. 23-26 illustrate schematic representations of holders for a device performing the scan according to the process of FIG. 22.

FIGS. 27 and 28 show schematic representations of an encapsulation of a network interface to facilitate the remote control of the process of FIG. 22.

FIGS. 29 and 30 illustrate schematic representations of encoding protocols to effectuate the remote controlling of the process of FIG. 22.

FIGS. 31 and 32 show schematic representations of graphical user interfaces to facilitate the remote controlling of the process of FIG. 22.

FIGS. 33 and 34 illustrate a schematic representation of a customized footwear insert provided by the system of FIG. 1.

FIG. 35 provides a table of preferred dimensions of various portions of the insert of FIGS. 33 and 34 according to a size of the user's foot determined in the process of FIG. 2.

FIGS. 36 and 37 show locations along which the height of the arch lines of the insert are calculated based on measured characteristics of the user's foot according to the process of FIG. 2.

FIGS. 38-41 illustrate tables for calculating or selecting the arch heights shown in FIGS. 36 and 37 according to the process of FIG. 2.

FIG. 42 shows ribs of different thicknesses for the footwear insert recommended according to the process of FIG. 2 according to a weight of the user.

### DETAILED DESCRIPTION OF THE PREFERRED EMBODIMENT

A foot-support selection or generation system in accordance with the principles of the invention is generally indicated at reference number 42 in the Figures of the attached drawings, wherein numbered elements in the Figures correspond to like numbered elements herein.

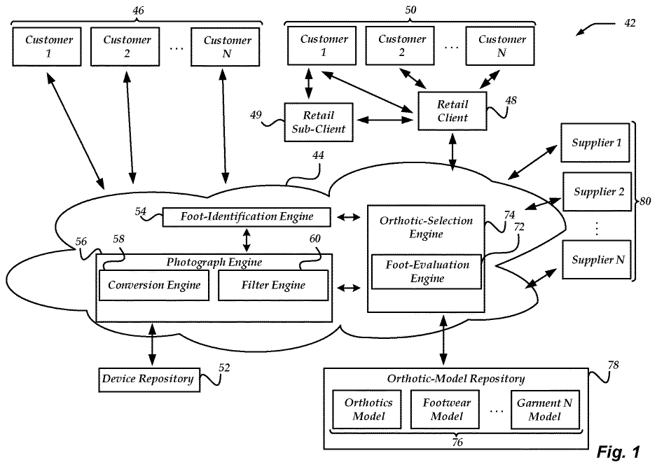

FIG. 1 is a functional block diagram that illustrates how the system 42 enables selecting or generating a foot-support orthotic, such as insoles, orthotics, orthopedics, or other footwear, based on as few as one photograph of a user's foot, preferably without placement of other objects in the photograph for scale identification. Many modern mobile devices enable users to unlock them or log into them with the users' biometrics instead of a password. Some of those mobile devices utilize photographs of users' faces as those biometrics. To prevent bypassing those security measures when an unauthorized user holds up a picture of an authorized user's face in front of the camera of the mobile device, the cameras of the mobile devices have been designed to make depth measurements with the photographs of the users' faces, such as the TrueDepth™ camera system in mobile phones or tablet devices from Apple Inc. A foot-support selection or generation computer 44 of the system 42 obtains such depth information with as few as a single photograph of a user's foot from such a mobile device. The computer 44 calculates from the depth information various measurements of the foot to determine a recommended foot-support orthotic for the user's foot. Herein the term orthotic may be switched with the term insole as the methods herein may be used for either item. The process of obtaining such data to enable the measurements and evaluation of the user's feet is therefore dramatically simplified by the system 42 compared to operation of specialized hardware, precise placement of objects for scale identification, or precise photographing/digitizing of the user's feet from predefined angles. Accordingly, the system 42 enables customers, such as the customers 46, to directly photograph their own feet without training, preferably without placement of other objects in the photograph for scale identification, and without transiting to a retailer with specialized hardware to scan or photograph the user's feet. The system 42 also therefore enables retailers, such as the retailer 48, to utilize its mobile devices or those of its employees to photograph the feet of its customers 50 instead of purchasing expensive, cumbersome, and large specialized equipment and without specialized training.

Preferably, the retailer client device 48 such as a mobile device or desktop computer is in wireless communication with a sub-client mobile device 49 that is positioned and oriented to take the photograph of the customer's foot in the retailer facility and under control of the retailer client device 48, and the sub-client 49 wirelessly provides the depth information and photograph to the retail client 48 to facilitate those devices 48, 49 respectively executing various portions of the processes described herein to select or generate a foot support.

In some embodiments, the computer 44 determines whether the mobile device is capable of providing depth information with a photograph by querying a device repository 52 based on the make or model of the mobile device. If the mobile device is not capable of providing such depth information, traditional methods of evaluating characteristics of the user's feet are performed to select or generate a foot-support orthotic. If the mobile device is capable of providing such depth information, the computer 44 causes the mobile device to display prompts or instructions to the user to position the mobile device in placing the user's foot within the field of view of the camera of the mobile device. A trained model, such as a foot-identification engine 54, preferably determines whether a foot is within the field of view of the camera of the mobile device. When the engine 54 determines that a foot is present in the field of view of the camera, a photograph engine 56 preferably obtains from the mobile device a digital photograph of the user's foot with depth information. A conversion engine 58 preferably converts the depth information into a measurable data set, such as a point cloud. A filter engine 60 preferably filters out all data from the data set except for the data related to the user's foot. A foot-evaluation engine 72 preferably generates various foot measurements from the filtered data set, such as foot length and arch height. An orthotic-selection engine 74 preferably evaluates the foot measurements against one or more parameters or models, such as orthotic models 76 in a orthotic-model repository 78. Based on the evaluation, the orthotic-selection engine 74 preferably provides to the user a recommendation for a foot-support orthotic that is determined to most closely match the characteristics of the user's foot or feet. Once the recommendation is confirmed by the user, the computer 44 preferably causes one of the suppliers 82 to select or generate (e.g., create through additive manufacturing or other methods) the recommended orthotic and provide it to the customer or retailer. In some cases, the retailer or a machine therein is one of the suppliers 80. Accordingly, the system 42 provides a simplified and faster approach for the customers 46, 50 to obtain customized selection or generation of foot-support orthotics.

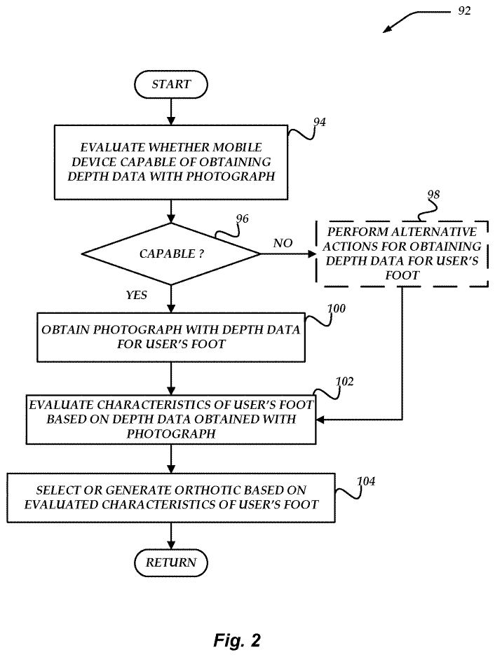

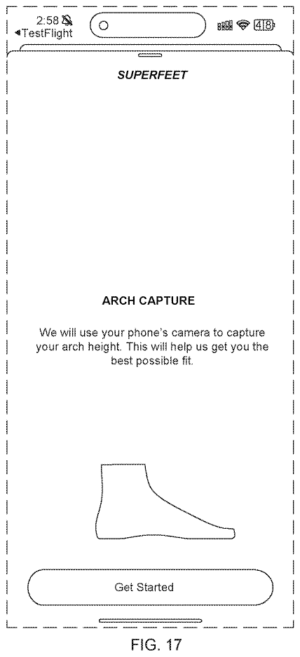

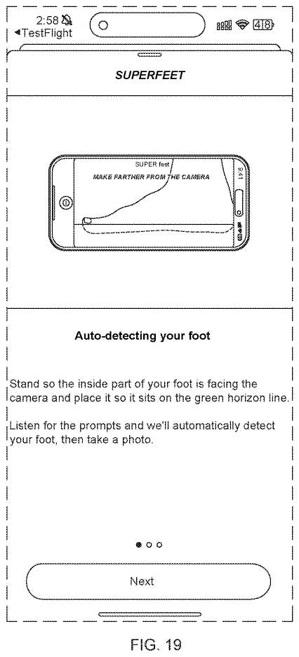

FIG. 2 shows a logical overview flow diagram that illustrates a foot-support selection or generation process 92 implemented, performed, or executed by the system 42 according to some embodiments. In some embodiments, one or more elements of the system 42 implement, perform, or execute one or more portions of the process 92, as discussed above with respect to FIG. 1. In some embodiments, the process 92 initiates at a start block responsive to a user accessing a website with the user's mobile device, initiating an application on the mobile device, or downloading another tool for executing software, such as an applet. In some embodiments, the user scans a quick-response code (i.e., a QR code) with the mobile device, and the QR code directs the user's mobile device to a website that enables the process 92 to be performed by the system 42 without the user's mobile device downloading an application. The user preferably provides permission for the computer 44 to interact with the camera of the mobile device through the website. In some embodiments, the user scans the QR code with the mobile device, and the QR code directs the user's mobile device to an App Clip from Apple Inc., an App Slice for Android devices, or another service that facilitates the mobile device executing instructions to facilitate performance of the process 92 without downloading a full application from an application store, such as Google Play or App Store from Apple Inc. In such embodiments, the App Clip, App Slice, or other such service facilitates the process 92 to be performed by the system 42 with the user's mobile device, and the user preferably provides permission for the computer 44 to interact with the camera of the mobile device through the App Clip, App Slice, or other such service. The display of the mobile device is preferably thereafter caused to display one or more of the interfaces shown in FIGS. 17-21. The process 92 is generally discussed with respect to a single user, single mobile device, and single foot for ease of understanding, yet the process 92 is executed in some embodiments with respect to both of the user's feet serially or in parallel to facilitate obtaining a foot-support orthotic for each foot.

In some embodiments, a retailer device client (e.g., device 48 in FIG. 1) has thereon a retailer-specific application that allows it to control the mobile device sub-client (e.g., device 49 in FIG. 1) that obtains the depth information with the photograph. In such embodiment, the retailer client and the retailer sub-client preferably respectively perform one or more of portions of the actions in the process 92 to in combination provide such process 92.

After the start block, at block 94, it is preferably determined whether the mobile device is capable of obtaining depth information with a photograph. Mobile devices that have depth sensors, such as the TrueDepth™ camera system in mobile phones or tablet devices from Apple Inc. or other camera systems with stereo vision, structured light systems, time of flight systems, or others that facilitate providing depth information with a single photograph are preferably considered capable at block 94. In some embodiments, the mobile device provides the computer 44 with an identification of the make and model of the mobile device (e.g., automatically responsive to a connection with the computer 44, manually by the user providing such information through a graphical user interface (GUI), or others), thereby enabling the computer 44 to determine whether such make or model is in a predefined list of capable devices.

At decision block 96, it is preferably determined whether the mobile device is capable of obtaining and providing depth information with a photograph, and if not, the process 92 preferably proceeds to optional block 98; otherwise, the process 92 preferably flows to block 100.

At block 98, a traditional process of determining an appropriate foot-support orthotic is executed because the user's device is not capable of obtaining and providing depth information with a photograph. An appropriate process includes a manual evaluation or one of those described in the Background of the Invention section herein. Block 98 is optional because the process may terminate or return to a calling process if it is determined at decision block 96 that the mobile device is not capable of obtaining depth information with a photograph. After block 98, the process 92 preferably flows to block 102.

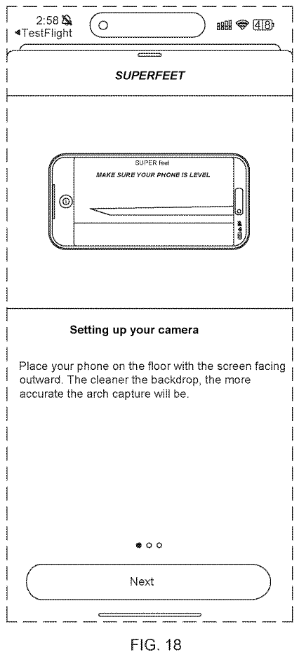

When it is determined that the user's mobile device is capable of obtaining and providing depth information with a photograph, the process flows to block 100. At block 100, the mobile device is caused to provide instructions to the user (see, e.g., FIGS. 18-20), and the mobile device takes a photograph of the user's foot with depth information of the user's foot. In some embodiments, the depth information is provided with the photograph to the computer 44 for evaluation.

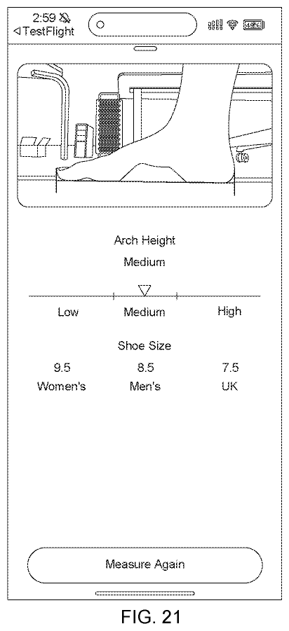

Next, at block 102, characteristics of the user's foot are evaluated based on the depth information obtained with the photograph. In some embodiments, the evaluated characteristics include the length of the foot, the length of the arch, the height of the arch, width of the foot, or others. In some embodiments, such characteristics are displayed on the user's mobile device (see FIG. 21).

After block 102, the process 92 proceeds to block 104 where a foot-support insole, such as an orthotic, is selected or generated based on the evaluated characteristics of the user's foot. In some embodiments, there is an inventory of various foot-support insoles that each have different characteristics, such as different lengths, different arch lengths, different arch heights, different widths, or others. In such embodiments, the repository 78 preferably has a register or database associating of each such insoles with an identifier for the insole, thereby enabling the computer 44 to select one of the insoles in the inventory with the characteristics that most closely match the evaluated characteristics of the user's foot. In other embodiments, the foot-support orthotic or insole is generated (e.g., manufactured with additive manufacturing techniques, such as 3D printing) to have characteristics that match or substantially match those of the user's foot.

In some embodiments, the process 92 continues operating until one or more events occur, such as purchase of the orthotic. In some embodiments, a recommendation for the selected or generated foot-support orthotic is provided to the user by displaying it on the mobile device with a GUI that enables the user to confirm whether to purchase the orthotic. Upon purchase by the user, the selected or generated orthotic is preferably provided to the user. Next, in some embodiments, control returns to a calling process.

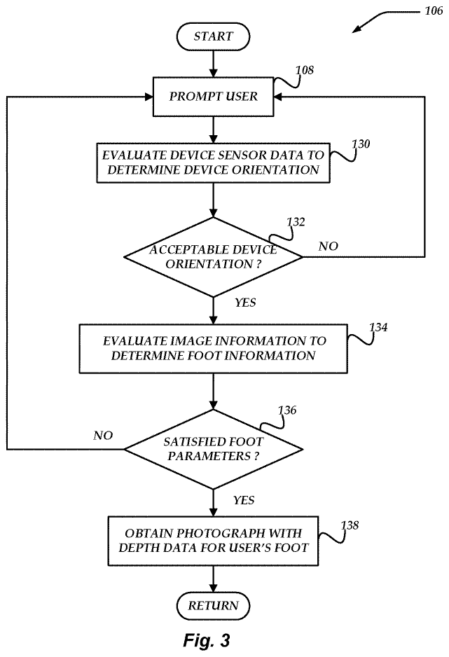

FIG. 3 illustrates a logical flowchart of a process 106 for obtaining the photograph with the depth information for the user's foot implemented, performed, or executed by the system 42 according to some embodiments. The process 106 is preferably executed as part of the block 100 of the process 92. In some embodiments, one or more elements of the system 42 (e.g., one of the mobile devices of the customers 46, the client device of the retailer 48, the retailer sub-client 49, the foot-identification engine 54, the photograph engine 56, or another component of the computer 44) implement, perform, or execute one or more portions of the process 106, as discussed above with respect to FIG. 1. In some embodiments, the process 106 initiates at a start block responsive to determining at the decision block 96 of the process 92 that the user's mobile device is capable of obtaining and providing depth information with a photograph, which preferably triggers the photograph engine 56 to engage with the mobile device.

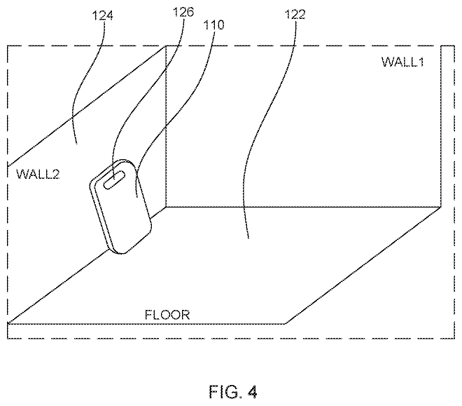

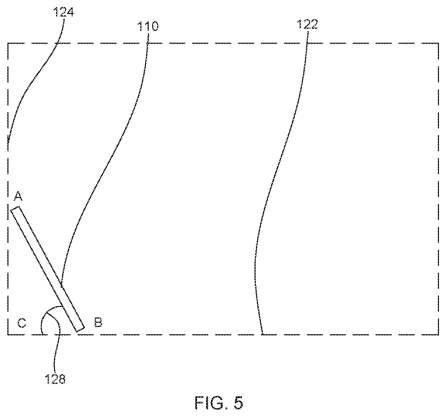

After the start block, at block 108, the user is preferably prompted to position the user's mobile device in a prescribed manner that enables the camera of the mobile device to capture a photograph of the user's foot, preferably with the display screen of the mobile device facing the user. Orienting the mobile device with the screen facing the user enables the user to follow prompts displayed on the screen, such as positioning the camera that has the depth-capture capability towards the user. As shown in FIG. 4, the user is preferably prompted to place the mobile device, such as a mobile smartphone 110, on the floor 122 on which the user will stand during the photographing of the user's foot. The user is also preferably prompted to lean the device 110 against a wall 124 such that the camera 126 of the mobile device 110 that is capable of obtaining depth information with a photograph facing away from the wall 124. In some embodiments, the user is prompted to orient the device 110 such that the angle 128 it makes with the floor 122 (angle ABC) is within a specified range, as shown in FIG. 5, such as at least approximately 77 degrees, to prevent unwanted depth information or visual distortion.

In the retail embodiment that includes the sub-client mobile device 49 of FIG. 1, the retailer client device preferably displays the prompts for an employee of the retailer 48 to articulate to the customer while the sub-client 49 is positioned and oriented (e.g., in a fixed stand) to image the customer's foot or feet. The sub-client 49 provides the data to the retailer client 48 to enable the client 48 to determine the appropriate prompt or provides an identification of the prompt to the retailer client 48 for display of such prompt.

Returning to FIG. 3, sensor data from the mobile device is preferably evaluated to determine the orientation of the mobile device at block 130. In some embodiments, the mobile device provides to the computer 44 information determined based on measurements by its one or more accelerometers or inclinometers, such as whether the mobile device is oriented in portrait or landscape mode, whether the mobile device is stable (i.e., resting on the floor 122), and the value of the angle 128 (or other information that permits calculating the angle 128). In some embodiments, such information is obtained with the Generic Sensor Application Programming Interface (API) for JavaScript® or a corresponding API for Swift code or script that facilitates obtaining such sensor information from mobile devices that access a website executing such script or an App Clip, App Slice, or other such service facilitating execution of one or more portions of the process 106. Such obtained information is preferably compared to predefined acceptable values, such as the mobile device being positioned in one of landscape or portrait mode rather than between such modes, in a stable stationary position rather than moving, and arranged with the angle 128 being at least 77 degrees. In other embodiments, the user manually confirms that the device is appropriately oriented and is stably stationed on the ground by interacting with a GUI that the computer 44 causes to be displayed on the mobile device.

At decision block 132, it is preferably determined whether the mobile device satisfies the parameters evaluated at block 130, and if not, the process 106 proceeds in some embodiments to continue prompting or to prompt the user again at block 108; otherwise, the process 106 preferably flows to block 134.

At block 134, a real-time evaluation of the field of view of the camera of the mobile device is preferably evaluated to determine certain foot information that indicates whether the user is ready for the photograph to be taken. In some embodiments, access to the camera of the mobile device is obtained with the AVFoundation framework's capture API from Apple Inc. through Swift code or script or the MediaDevices API for JavaScript® that facilitates obtaining such image information from mobile devices that access a website executing such script or an App Clip, App Slice, or other such service facilitating execution of one or more portions of the process 106.

At decision block 136, it is determined in some embodiments whether the foot parameters evaluated at block 134 are satisfied, and if not, the process 106 preferably proceeds to continue prompting or to prompt the user again at block 108; otherwise, the process 106 preferably flows to block 138.

At block 138, a photograph with depth information for the user's foot is obtained from the mobile device. In some embodiments, the photograph is obtained with the AVFoundation framework's capture API from Apple Inc. through Swift code or script or the MediaDevices API for JavaScript® that facilitates obtaining such photograph with such depth information from mobile devices that access a website executing such script or an App Clip, App Slice, or other such service facilitating execution of one or more portions of the process 106.

In some embodiments, the process 106 continues operating until one or more events occur, such as a photograph for both of the user's feet being obtained or the user approving the one or more photographs. Next, in some embodiments, control returns to a calling process, such as the process 92.

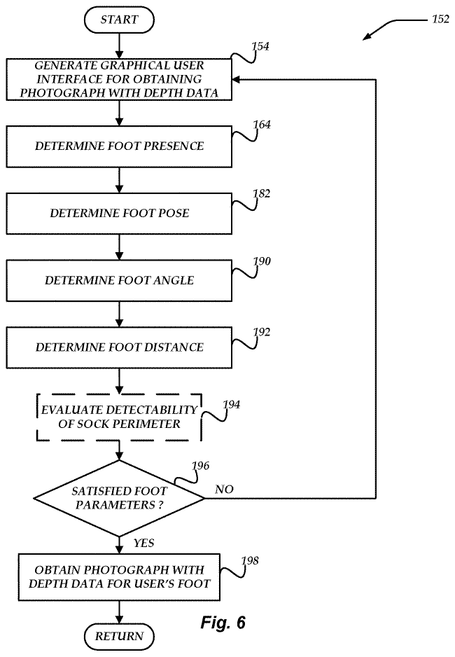

FIG. 6 illustrates a logical flow diagram of a process 152 for determining whether the user is ready for the photograph of the user's foot to be taken. The process 152 is implemented, performed, or executed by the system 42 according to some embodiments. The process 152 is preferably executed as part of the blocks 134 and 136 of the process 106. In some embodiments, one or more elements of the system 42 (e.g., one of the mobile devices of the engine 54, the photograph engine 56, or another component of the computer 44, such as a device of one or more of the suppliers 80) implement, perform, or execute one or more portions of the process 152, as discussed above with respect to FIG. 1. In some embodiments, the process 152 initiates at a start block responsive to determining at the decision block 132 of the process 106 that the user's mobile device is appropriately oriented in a stationary manner, which preferably triggers the photograph engine 56 to engage with camera of the mobile device. In the retail embodiment that includes the sub-client 49, it is the camera of the sub-client 49.

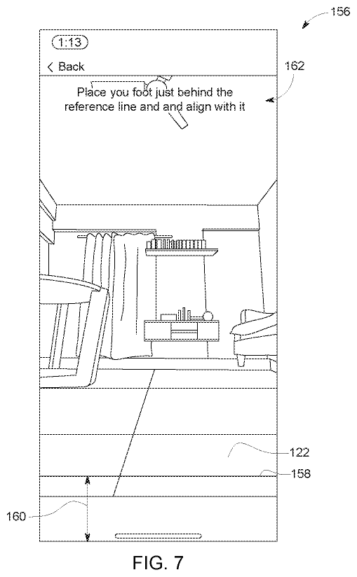

After the start block, at block 154, a GUI is caused to be displayed on the mobile device for obtaining the photograph with the depth data for the user's foot. In the retail embodiment that includes the sub-client 49, the GUI is shown on at least the display of the client device of the retailer 48 such as a tablet (e.g., an iPad) or a desktop computer in communication with the sub-client 49. As shown in FIG. 7, the computer 44 preferably causes the mobile device to display the real-time image obtained by the camera of the mobile device along with the GUI components superimposed thereon. In the retail embodiment that includes the sub-client 49, it is preferably shown on at least the display of the client device of the retailer 48. In some embodiments, a different set of information is displayed on the client device of the retailer 48, such as the status of the sub-client 49 and available commands for the retailer employee to select.

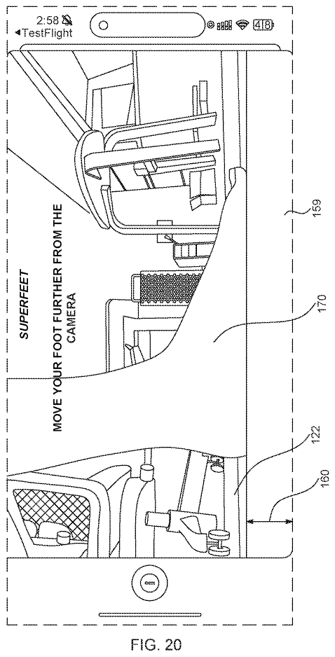

It is preferably determined where in the image provided by the camera is the desired range for the user to place the user's foot. Mobile devices configured to use depth information of the user's face for security purposes are typically optimized to capture such depth information in the typical range of distances at which most users hold their mobile device from their face. In some embodiments, the accuracy of depth information by certain mobile devices, such as mobile devices implementing the TrueDepth™ camera system from Apple Inc., is increased in the distance range of approximately 300 mm to 500 mm. Accordingly, a GUI 156 (see FIG. 7) preferably superimposes a reference indication, such as a reference line 158 (or a reference region 159 as shown in FIG. 20), at a distance 160 from the bottom of the screen of the mobile device that superimposes the indication 158 or 159 on the image of a portion of the floor 122 that corresponds to such increased-accuracy distance from the camera. In some embodiments, the reference indicator 158 is superimposed on the image of a portion of the floor 122 that is approximately 400 mm from the camera when the mobile device is in portrait mode with the camera positioned closer to the edge of the mobile device that is not touching the floor 122. In some embodiments, the reference indicator 158 is superimposed on a portion of the floor 122 that is approximately 300 mm from the camera when the mobile device is in landscape mode. Accordingly, such distance or range that has been determined to be appropriate for placement of the user's foot relative to the camera of the mobile device is also where the GUI displays artifacts for the user to follow to facilitate increasing scanning quality.

In some embodiments, the reference indicator 158 or 159 is superimposed on a portion of the floor 122 that is farther from the camera when the mobile device is in portrait mode than when it is in the landscape mode because, in the portrait mode, the field of view of the camera is narrower left to right than when the mobile device is in landscape mode, thereby increasing the likelihood that the entire foot will be captured in the field of view of the camera when the foot is placed slightly farther away from the camera. In some embodiments, the GUI 156 also displays or the mobile device audibly provides a prompt 162 instructing the user to place the user's foot on or slightly behind the reference indicator 158 while aligning the foot with it. In the retail embodiment that includes the sub-client 49, the prompt 162 is preferably displayed on the client device 48 to facilitate the employee of the retailer communicating the instruction to the customer. In some embodiments, the reference indicator 158 or the customer's foot are displayed on the client device 48.

In some embodiments, such position of the reference indicator 158 or 159 is determined dynamically in real-time by streaming the depth information from the camera of the mobile device with the live image obtained by the camera in real time. The depth information typically indicates a distance of a pixel from the front-facing camera of the mobile device and, in some embodiments, is obtained in real time with the AVFoundation framework's capture API to read data from the TrueDepth camera provided by Apple Inc. (e.g., creating an AVCaputreSession and outputting the depth data with AVCaptureDepthDataOutput) in real-time. In some embodiments, such session enables capturing motion of the mobile device, such as with CMMotionManager, including the device stability, orientation, landscape or portrait mode, or others. In some embodiments, AVCaptureDataOutputSynchronizer is implemented to coordinate time-match delivery of the data obtained with AVFoundation and CMMotionManager. Based on the depth information obtained for each pixel output by the camera, the display of the mobile device is caused to display the reference indicator 158 or 159 at the selected distance (e.g., approximately 300 mm or 400 mm from the camera depending on whether the mobile device is in portrait or landscape mode).

In some embodiments, the position to display the reference indicator 158 or 159 is determined based on a point cloud generated from the depth information, rather than directly from the depth information. The depth information preferably provides a depth map based on the photograph or streamed image obtained by the camera. The depth map is typically defined in the image space (i.e., an image coordinate system) with point identifications relative to the top left corner of the photograph and expressed in pixels instead of distances. In particular, tools that rely on depth information from mobile devices so enabled, such as FaceID using the TrueDepth™ camera system in mobile phones or tablet devices from Apple Inc., are typically not producing measurements and instead merely compare the depth information to expected results to determine whether the authorized biometrics are present. Accordingly, the depth information itself or its depth map are typically not particularly helpful in measurements. The depth map of the depth information is therefore preferably converted into a point cloud by converting the image space data into object space data (i.e., an object coordinate system), in which the coordinate system identifies distances rather than pixels. Such conversion is preferably performed with known trigonometric formulas and in some embodiments the intrinsics matrix provided by Apple Inc. for developers to convert between a two-dimensional camera plane and a three-dimensional world coordinate space.

In other embodiments, the location to display the reference indicator 158 or 159 is based on the height and angle of the camera of the mobile device. In such embodiments, it is preferably determined which edge of the mobile device is in contact with the floor 122 when the orientation of the mobile device is evaluated in block 130 of process 106 (see FIG. 3). The device repository 52 (see FIG. 1) preferably indicates the distance between each edge of the mobile device and its camera, thereby facilitating obtaining such distance between the camera and the edge in contact with the floor 122 by querying the repository 52 based on the mobile device model in the orientation of the mobile device. Accordingly, based on the obtained distance between the camera and the edge of the mobile device that is determined to be in contact with the floor 122 and the angle 128 (see FIG. 5), the height of the camera of the mobile device from the floor 122 is preferably determined. In particular, the height of the camera is preferably determined as being equal to the sine of angle 128 (i.e., sin (angle 128)) divided by the distance between the camera and the edge of the mobile device that is determined to be in contact with the floor 122.

In some embodiments, the plane of the floor 122 is determined based on the three-dimensional point cloud with a least square plane fitting algorithm. The selected distance for the reference indicator 158 is preferably identified on the floor plane. The reference indicator 158 is preferably plotted in the three-dimensional point cloud at the selected distance. The plotted reference indicator 158 is preferably converted from the three-dimensional point cloud into the two-dimensional camera plane (i.e., an image coordinate system) and superimposed on the display of the mobile device to provide a dynamic display of the reference indicator 158 in real-time.

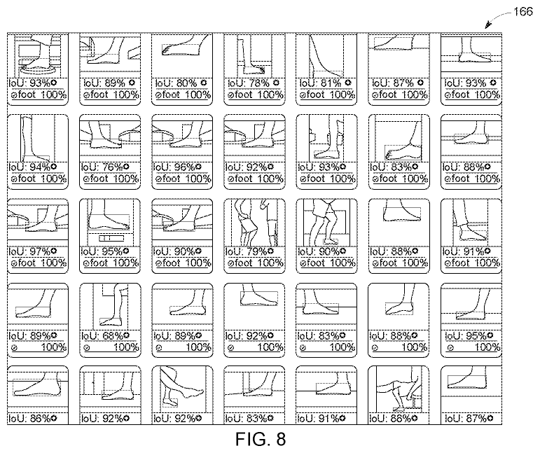

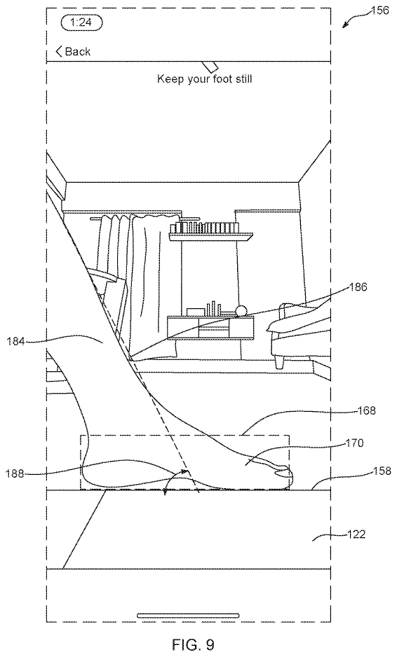

Next, at block 164, it is preferably determined whether the user's foot is present in the field of view of the camera. In some embodiments, such determination is made by a model (e.g., machine learning models, deep learning models, or others) that has been trained on a prescribed quantity of photographs to identify feet, such as the foot-identification engine 54 (see FIG. 1). Such model preferably executes on the user's mobile device to facilitate detecting the user's foot even while the user's mobile device is offline and without consuming resources outside of the user's mobile device. FIG. 8 shows a sampling 166 of the training data set including such photographs of feet on which the foot-identification engine 54 is trained for foot detection. The GUI 156 preferably shows one or more graphics, such as a rectangle, superimposed on each image of a foot, with the one or more graphics indicating the front and rear edges in the bottom-most edges of the user's foot. Accordingly, as shown in FIGS. 8 and 9, a rectangle 168 is superimposed on the image of the user's foot 170 in the field of view of the camera of the user's mobile device. One short edge of the rectangle 168 preferably identifies the heel edge of the user's foot while the opposite shore edge of the rectangle 168 preferably identifies the toe edge of the user's foot, while the bottom long edge of the rectangle preferably touches the lowest points of the user's foot in the image. When a foot is identified, the reference indicator 158 or 159 or another aspect of the GUI preferably provides positive feedback to the user, such as changing the color of the reference indicator 158 or 159 from red to green.

After identifying the user's foot, the process 152 (FIG. 6) proceeds to block 182 at which the pose of the foot is determined. In some embodiments, the pose of the foot 170 is determined based on whether the foot 170 is weight-bearing. The arch height of the user's foot 170 is typically lower when the foot 170 is weight-bearing. In some embodiments, the GUI instructs the user to make the foot 170 non-weight-bearing (e.g., displaying a prompt for a retail employee to communicate to the customer). In other embodiments, the GUI executes one or more of the processes described herein when the foot 170 is weight-bearing and again when the foot 170 is non-weight-bearing to facilitate selecting or generating a foot-support orthotic based on information obtained from both situations.

In some embodiments, it is determined whether the foot 170 is weight-bearing based on whether the leg 184 having the foot 170 is substantially vertical. Such determination is preferably made by evaluating whether a front edge 186 of the leg 184 provides an angle 188 that is substantially orthogonal to the floor 122 or indication line 158 or region 159. In some embodiments, when the angle 188 is less than 90 degrees by more than a predefined threshold (e.g., approximately 5 degrees), it is determined that the foot 170 is not weight-bearing. In other embodiments, user input, such as manual manipulation of the GUI or voice commands, are used to determine whether the user indicates that the user is placing weight on the foot 170.

At block 190, the angle of the foot 170 relative to the camera is determined. It is preferably determined whether the arch side of the foot 170 (i.e., the medial side of the foot 170) faces the camera. In some embodiments, whether the length of the foot 170 is generally parallel to the reference indication 158 or 159. As shown in FIG. 9, the bottom of the rectangle 168 is generally parallel to the reference line 158 or 159, thereby indicating that the foot 170 is generally parallel to the reference indication 158 or 159.

At block 192, the distance between the camera and the foot 170 is determined. In some embodiments, the depth information for the foot 170 has not yet been obtained from the mobile device. Accordingly, in such embodiments, the distance between the camera and the foot 170 is determined based on the proximity of the bottom of the foot 172 the reference indication 158 or 159. As shown in FIG. 9, the bottom of the rectangle 168 is above the reference indication 158 or 159 distance measurable by the number of pixels between the reference indication 158 or 159 in the bottom of the rectangle 168.

At optional block 194, it is determined whether the perimeter of a sock worn by the foot in the field of view of the camera is detectable. Some socks are fuzzier than others. Highly fuzzy socks have perimeters that are difficult to accurately detect. Black socks also have perimeters that are difficult to accurately detect. Accordingly, it is optional for whether the user will be allowed to proceed while wearing socks, but in those embodiments in which the user is permitted to wear socks, a determination is preferably made as to whether the perimeter of the sock is detected.

At decision block 196, it is determined in some embodiments whether parameters for the foot are sufficiently satisfied by the values determined at the blocks 164, 182, 190, 192, and optionally 194, and if not, the process 152 preferably returns to the block 154 to continue evaluating whether such parameters are satisfied; otherwise, the process 152 preferably flows to the block 198. The parameters are preferably predefined threshold values against which the determined values are compared. Whether the foot is present is preferably a binary value (e.g., true or false) that is true to satisfy the parameter associated with the block 164. Whether the foot is weight-bearing is preferably also a binary value that is false to satisfy the parameter associated with the block 182. Whether the medial side of the foot faces the camera is preferably a binary value that is true to satisfy the corresponding parameter associated with the block 190. The angle of the foot relative to the camera is preferably a numerical value that is within approximately 1, 2, 3, 4, 5, 7.5, or 10 degrees of parallel to the horizontal reference line 158 or region 159 to satisfy the corresponding parameter associated with the block 190. The distance from the foot to the camera is preferably also a numerical value that is within a predefined range, such as approximately 300 mm to 500 mm, to satisfy the parameter associated with the block 192. In some embodiments, such parameter is satisfied when the foot is within such range and on or above the reference line 158 or region 159. In some embodiments, the distance between the camera and the foot is estimated based on the number of pixels between the reference line 158 or region 159 in the bottom of the rectangle 170, with each row of pixels being estimated at a certain number of millimeters. Whether the perimeter of a sock is detectable is preferably a binary value that is true to satisfy the parameter associated with block 194.

At block 198, with preferably all the parameters for the foot satisfied, a photograph with depth information for the users foot is obtained from the mobile device.

In some embodiments, the process 152 continues operating until one or more events occur, such as a photograph for both of the user's feet being obtained or the user approving the one or more photographs. Next, in some embodiments, control returns to a calling process, such as the process 92.

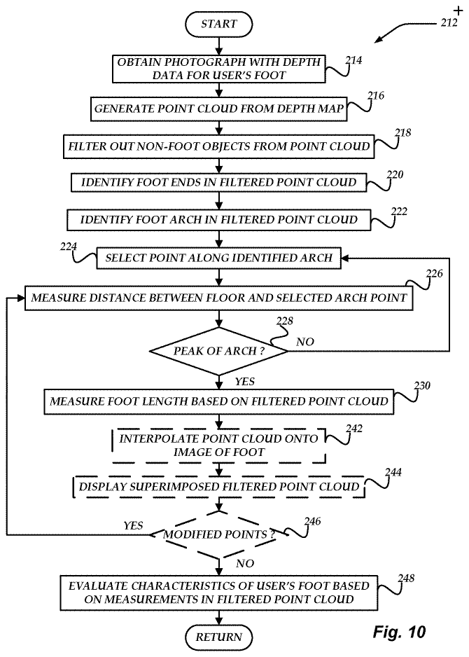

FIG. 10 illustrates a logical flowchart of a process 212 for evaluating characteristics of the user's foot based on a photograph obtained with depth information for the user's foot. The process 212 is preferably implemented, performed, or executed by the system 42. The process 212 is preferably executed as part of the block 102 of the process 92. In some embodiments, one or more elements of the system 42 (e.g., one of the mobile devices of the engine 54, the photograph engine 56, the conversion engine 58, the filter engine 60, the foot-evaluation engine 72, or another component of the computer 44, such as a device of one or more of the suppliers 80) implement, perform, or execute one or more portions of the process 212, as discussed above with respect to FIG. 1. In some embodiments, the process 212 initiates at a start block responsive to the foot parameters being satisfied at the block 196 of the process 152 or the user manually indicating that the photograph of the user's foot should be taken, such as by providing a verbal command or providing a touch input on the GUI displayed on the user's mobile device.

After the start block, at block 214, the photograph of the user's foot with depth information for the user's foot is obtained from the user's mobile device. In the retail embodiment that includes the sub-client 49, the photograph with the depth information is obtained from the sub-client device 49.

At block 216, a depth map is preferably obtained from the depth information obtained with the photograph. For the reasons discussed regarding the block 154 of the process 152, the depth information and the depth map are typically not particularly helpful as provided from the mobile device for measuring characteristics of the user's foot. The depth map is preferably converted from the image space (i.e., a two-dimensional camera plane with an image coordinate system) to the object space (i.e., a three-dimensional world space with an object coordinate system) to provide a three-dimensional point cloud in the same manner as described regarding the block 154 of the process 152.

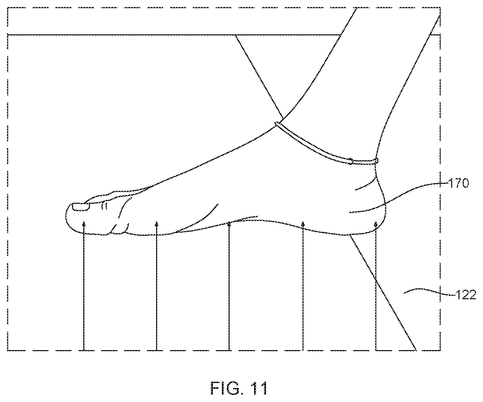

At block 218, the non-foot objects are filtered out from the point cloud. Prior to such filtering, the point cloud is preferably vertically scanned to identify the distance of the foot from the camera, as schematically represented by the vertical arrows in FIG. 11 superimposed on a photograph of the foot 170 for ease of understanding. Based on the vertical scan, a height value (i.e., H value) is assigned to each point relative to the floor plane. In some embodiments, the floor plane has already been identified in the point cloud, such as at the block 154 in the process 152. In other embodiments, the floor plane is re-identified in the same manner as described for the block 154 based on the point cloud generated from the depth map obtained at the block 216. At the bottom of FIG. 11, the H value of each point is approximately zero. Where the H values of the points in the point cloud begin increasing is where the foot is preferably determined to be placed. If no such foot is identified, the user is preferably instructed through the mobile device to repeat the process of capturing the photograph of the foot with the foot in the field of view of the camera. If the foot is identified at a distance greater than a threshold from the camera, such as approximately 500 mm when the mobile device is in landscape mode or approximately 550 mm when the mobile device is in portrait mode, the user is instructed to repeat the process of capturing the photograph of the foot with the foot within the prescribed distance range of the camera. If the foot is identified at a distance less than threshold from the camera, such as approximately 250 mm when the mobile device is in landscape mode or approximately 300 mm when the mobile device is in portrait mode, the user is also instructed to repeat the process of capturing the photograph of the foot with the foot within the prescribed distance range of the camera. If the foot is identified within the prescribed distance range, such as between approximately 250 and 500 mm when the mobile devices in landscape mode or between approximately 300 mm and 550 mm when the mobile devices in portrait mode, the determined distance between the foot in the camera is approved.

With the foot distance determined to satisfy the threshold parameters, the filtering preferably involves a series of one or more filters, such as one or more of a depth filter, a floor filter, height filter, precision depth filter, cluster filter, irregularity perimeter filter, or others. In some embodiments, the depth filter removes from the three-dimensional point cloud all points with a distance greater than approximately 700 mm from the camera because the foot is expected to be at a distance of approximately 250 mm to 500 mm.

The floor filter preferably removes each point that is part of the floor. The distance between the floor plane and each point remaining in the point cloud after the depth filtering is preferably compared to a threshold distance (e.g., a plane inlier threshold), such as approximately 4 mm, to determine whether the point is an inlier and therefore treated as a point on the plane (i.e., a part of the floor). All such inliers are filtered out by the floor filter to remove the floor plane from the three-dimensional point cloud.

The height filter preferably removes from the three-dimensional point cloud each point with a distance from the floor plane greater than a height threshold, such as approximately 70 mm, because any portion of the user's body above such height is typically immaterial to selecting or generating a foot-support orthotic.

The precise depth filter preferably determines the median depth position (i.e. the Z value) of the remaining points in the three-dimensional point cloud and removes all points that are farther than a depth threshold from such determined median depth, such as all remaining points that are farther than approximately 70 mm from the determined median depth. In particular, points closer to the camera than the median depth by more than the depth threshold or farther from the camera than the median depth by more than the depth threshold are probably not part of the foot.

The cluster filter preferably determines the largest cluster of points remaining in the point cloud, such as with a density-based clustering algorithm (e.g., DBSCAN), and removes from the three-dimensional point cloud all remaining points that are not part of the determined cluster because they are likely not part of the foot.

The irregularity perimeter filter preferably smooths the perimeter of the remaining points in the three-dimensional point cloud. In some embodiments, the perimeter filter smooths the perimeter of the entire foot in the point cloud. In other embodiments, the perimeter filter smooths the perimeter of only one or both of the toe end portion of the foot or the heel end portion of the foot in the point cloud. The perimeter filter preferably identifies deviations in depth values in the perimeter points of the point cloud and removes or adds points as appropriate to decrease the deviations and smooth the perimeter of the foot in the point cloud. In some embodiments, a deviation is identified as requiring smoothing when the deviation exceeds a deviation threshold, such as approximately 1, 2, 5, or more millimeters, compared to the adjacent points.

At block 220, the ends of the foot (the toe end and the heel end) are preferably identified in the filtered three-dimensional point cloud, which are preferably identified as the leftmost and rightmost points in the point cloud. The toe end is preferably identified as having a lower density of points compared to the heel end, thereby facilitating identifying whether the left or right end is the toe end and whether the other is the heel end.

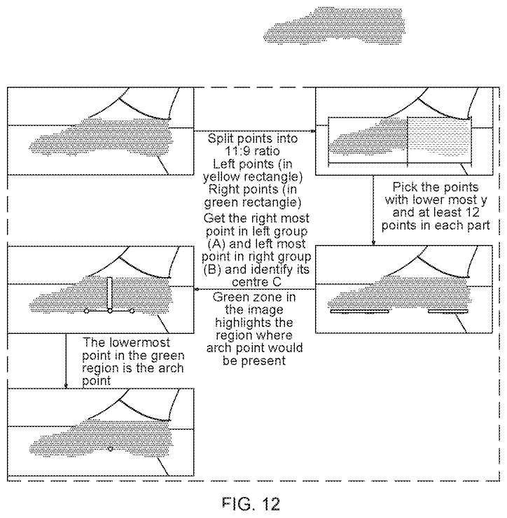

At block 222, the arch of the foot is identified in the filtered point cloud. In FIG. 12, the filtered point cloud is shown superimposed on the photograph of the foot for ease of understanding. The top left image schematically represents the filtered point cloud. The top right image shows the point cloud split into two portions. In some embodiments, the two portions have nonequal lengths (i.e., the dimension measured along the x-axis), such as having a ratio of lengths of 11:9 with the toe end portion having the greater of the two lengths compared to the heel end portion. As shown in FIG. 12, the toe end portion is on the left and the heel end portion is on the right.

The right middle image of FIG. 12 represents identifying points in each of the toe end portion and the heel end portion that have the lowest vertical values (i.e., the dimension measured along the y-axis). If such identification results in fewer than 12 points in the point cloud in one or both of the toe end portion or the foot end portion, the vertical or Y value is increased (e.g., increase the Y value by approximately 1 mm). Such identification of the lowest points having the Y value and increasing the Y value preferably repeats until at least 12 points in the point cloud in each of the toe end portion and the foot end portion are identified.

Next, as shown in the left middle image in FIG. 12, the front and rear edges of the arch are preferably identified. The point in the left portion of the point cloud (i.e., the toe end portion in FIG. 12) immediately to the right of the points having the most recently selected Y value and having a greater Y value that such selected Y value is preferably determined to be the left edge of the arch. The point in the right portion of the point cloud (i.e., the heel and portion in FIG. 12) immediately to the left of the points having the most recently selected Y value and having a greater Y value that such selected Y value is preferably determined to be the right edge of the arch.

At block 224, each point is preferably selected along the bottom perimeter of identified arch to facilitate determining whether the selected point corresponds to the maximum height of the arch (i.e., the largest vertical or Y value).

At block 226, the vertical or Y value of each selected point is preferably determined.

At decision block 228, it is preferably determined whether the selected point is the highest point along the bottom perimeter of the identified arch, and if not, the process 212 in some embodiments returns to the block 224 to continue searching for the highest point along the perimeter of the arch; otherwise, the process 212 proceeds to block 230.

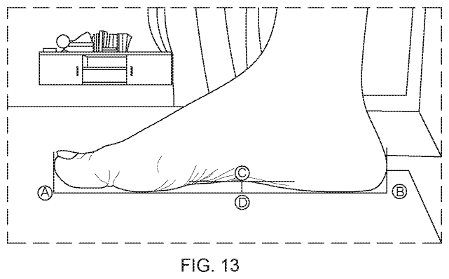

By block 230, the elevation of the points along the bottom perimeter of the arch in the filtered point cloud have been evaluated as shown in the left middle image of FIG. 12 to identify the highest point along the bottom perimeter of the arch as shown in the bottom left image of FIG. 12. Accordingly, the height of the arch has been determined in the point cloud (see distance CD in FIG. 13). At the block 230, the length of the foot is preferably determined in the point cloud (see distance AB in FIG. 13).

At optional block 242, the filtered point cloud is preferably interpolated onto the photograph of the foot. In other embodiments, the measurements of the user's foot characteristics are interpolated onto the photograph of the foot, such as one or more of the length AB of the foot, the height CD of the arch, the length of the arch, or others.

At optional block 244, the interpolated image (e.g., one or more of the filtered point cloud superimposed on the photograph of the foot or a representation of such point cloud, the user's foot characteristics superimposed on the photograph of the foot, or others) is preferably displayed on the user's mobile device (see FIG. 21).

At optional decision block 246, it is preferably determined whether the user modified any points or values displayed on the user's device to correct or any just any measurements or determinations regarding the user's foot or its characteristics, and if not, the process 212 preferably proceeds to block 248; otherwise, the process 212 preferably returns to the block 226 to repeat the measurements of the process 212 based on the modifications provided by the user. The blocks 242, 244, and 246 are optional because the system 42 in some embodiments does not provide the user with the opportunity to modify the point cloud or the measured values. In other embodiments, the GUI facilitates the user replacing or otherwise modifying the measured values (e.g., typing new values, incrementing or decreasing the values, or others), such as with text boxes or selectable arrows. In some embodiments, the GUI facilitates the user modifying the point cloud or determinations related to it, such as adjusting the parameters of the foot, the perimeter of the arch, the endpoints of the foot, the endpoints of the arch, or the location of the high point of the arch (e.g., draggable boundaries, points, or others). In some such embodiments, the GUI enables the user to move points directly on the displayed image to remove mistakes or outliers, such as with a 3D TOUCH or 3D POINTER interface, such as by dragging the left or right ends shown by the vertical lines at the toe end and heel end of the foot in FIG. 21 or by dragging one or more points on the superimposed representation of the point cloud shown in FIG. 21.

At block 248, the determined characteristics of the user's foot (e.g., the length AB of the foot, the height CD of the arch, the length of the arch, or others) are preferably evaluated to facilitate determining a recommendation of a foot-support orthotic for the user's foot.

In some embodiments, the process 212 continues operating until one or more events occur, such as the user approving the recommendation of the foot-support orthotic, both feet of the user having been evaluated, or others. Next, in some embodiments, control returns to a calling process, such as the process 92.

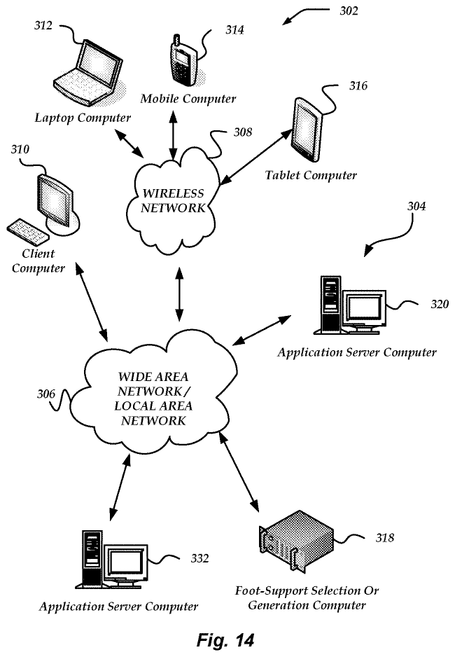

FIG. 14 shows components of an environment 302 in which a system 304 of the invention, such as the system 42 of FIG. 1, is practiced according to some embodiments. As shown, the environment 302 of FIG. 14 includes local area networks (LANs) or wide area networks (WANs) 306, wireless network 308, client computers 310, 312, 314, 316, foot-support selection or generation computer 318, application server computer 320, application server computer 332, or others.

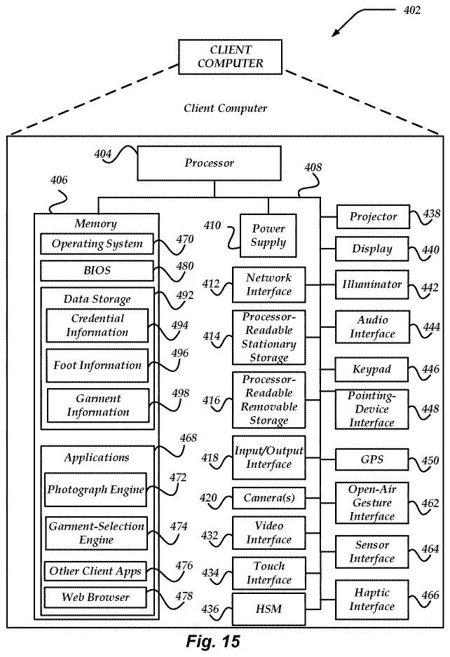

An embodiment of the client computers 310-316 is described with respect to FIG. 15. In some embodiments, one or more of the client computers 310-316 operate over one or more wired or wireless networks, such as the networks 306, 308. In some embodiments, one or more of the client computers 310-316 are configured to operate within a business or other entity to perform a variety of services for the business or other entity. In some embodiments, one or more of the client computers 310-316 are implemented as or executing respective ones of the customers 46, retailers 48, or retailer sub-clients 49 of FIG. 1. In some embodiments, one or more of the client computers 310-316 are operated by respective constituents of the customer 46 or retailers 48. In some embodiments, one or more of the computers 318, 320, 332 are implemented as the computer 44 or one or more of its engines. In some embodiments, the client computers 310-316 include one or more other client applications that are configured to facilitate providing information or receiving information from the engines of FIG. 1, such as receiving and granting permission requests from one or more of the computers 318, 320, 332 for access to hardware of the client computers 310-316, providing images or photographs with depth information from the client computers 301-316 to one or more of the computers 318, 320, 332, and receiving and displaying the GUI provided by one or more of the computers 318, 320, 332.

In some embodiments, one or more of the client computers 310-316 are web enabled. A web-enabled client computer preferably includes a browser application, which in the context of this application is configured to send requests and receive responses over the web. The browser application is preferably configured to receive or display graphics, text, multimedia, or others, preferably employing virtually a web-based language. In some embodiments, the browser application is enabled to employ JavaScript, HyperText Markup Language (HTML), extensible Markup Language (XML), JavaScript Object Notation (JSON), Cascading Style Sheets (CSS), or others to display or send a message. In some embodiments, a user of such client computer may employ the browser application to perform various activities over a network (e.g., online). However, another application is used to perform various online activities in some embodiments.

The client computers 310-316 also preferably include one or more other client applications that are configured to receive or send content from or to another computer. The client application preferably has a capability to send or receive content or other information or signals. The client application preferably provides information that identifies itself or the client computer, including a type, capability, name, or others. In some embodiments, the client computers 310-316 uniquely identify themselves through one or more of a variety of mechanisms, including an Internet Protocol (IP) address, a phone number, Mobile Identification Number (MIN), an electronic serial number (ESN), a Media Access Control (MAC) address, a client certificate, or other device identifier. Such information is preferably provided by the client computer in one or more network packets or other collections of data, which are preferably sent between client computers, the foot-support selection or generation computer 318, the application server computer 320, the application server computer 332, or other computers.

The client computers 310-316 are preferably further configured to include a client application that enables an end user to log into an end-user account that is in some embodiments managed by another computer, such as the foot-support selection or generation computer 318, the application server computer 320, the application server computer 332, or others. Such an end-user account, in some embodiments, enables the end user to manage one or more online activities, which preferably includes project management, software development, system administration, configuration management, search activities, social networking activities, various website browsing, communication with other users, or others. Further, the client computers 310-316 are preferably arranged to enable users to provide configuration information or other information discussed regarding the processes described herein to one or more of the foot-support selection or generation computer 318, the application server computer 320, or the application server computer 332. Also, the client computers 310-316 are preferably arranged to enable users to display reports, interactive user-interfaces, results or other information described herein provided by one or more of the foot-support selection or generation computer 318, the application server computer 320, or the application server computer 332.

In some embodiments, the wireless network 308 is configured to couple the client computers 310-316 and its components with the network 306 or other wireless networks. The wireless network 308 in some embodiments includes one or more wireless networks or sub-networks that in some embodiments further overlay stand-alone ad-hoc networks or others to provide an infrastructure-oriented connection for the client computers 310-316. Such sub-networks in some embodiments include mesh networks, Wireless LAN (WLANs), cellular networks, or others.

The wireless network 308 in some embodiments further includes an autonomous system of terminals, gateways, routers, or others connected by wireless radio links or others. These connectors are preferably configured to move freely and randomly and to organize themselves arbitrarily, such that the topology of wireless network 308 in some embodiments changes rapidly.

The wireless network 308 in some embodiments employs one or more access technologies including 2nd (2G), 3rd (3G), 4th (4G), 5th (5G) generation radio access for cellular systems, WLAN, Wireless Router (WR) mesh, or others. The access networks in some embodiments enable wide area coverage for mobile computers, such as the client computers 310-316 with various degrees of mobility. In some embodiments, the wireless network 308 enables a radio connection through a radio network access such as Global System for Mobile communication (GSM), General Packet Radio Services (GPRS), Enhanced Data rates for GSM Evolution (EDGE), code division multiple access (CDMA), time division multiple access (TDMA), Wideband Code Division Multiple Access (WCDMA), High Speed Downlink Packet Access (HSDPA), Long Term Evolution (LTE), or others. Accordingly, the wireless network 308 preferably enables information to travel between one or more of the client computers 310-316 and one or more other computers, networks, cloud instances, or others.

The network 306 is preferably configured to couple one or more networked computers with one or more other computers, including one or more of the foot-support selection or generation computer 318, the application server computer 320, the application server computer 332, one or more of the client computers 310-316, or others. The network 306 is preferably configured to implement computer readable media for communicating information from one electronic device to another. Also, the network 306 includes in some embodiments the Internet in addition to local area networks (LANs), wide area networks (WANs), or direct connections, such as through a universal serial bus (USB) port, Ethernet port, or other forms of computer-readable media. On an interconnected set of LANs, including those based on differing architectures or protocols, a router acts preferably as a link between LANs, enabling messages to be sent from one to another. In addition, communication links within LANs in some embodiments includes twisted pair, coaxial cable, fiber optics, wave guides, and other wired media and wireless media such as acoustic, RF, infrared, or other wireless media, while communication links between networks in some embodiments utilize analog telephone lines, full or fractional dedicated digital lines including T1, T2, T3, and T4, or other carrier mechanisms including E-carriers, Integrated Services Digital Networks (ISDNs), Digital Subscriber Lines (DSLs), wireless links including satellite links, or other communications links. Moreover, communication links in some embodiments employ digital signaling technologies, including, for example, DS-0, DS-1, DS-2, DS-3, DS-4, OC-3, OC-12, OC-48, or others. In some embodiments, remote computers and other related electronic devices are remotely connected to either LANs or WANs of the network 306 via a modem and temporary telephone link. Additionally, the communication media of the network 306 preferably carries computer readable instructions, data structures, or program modules that include the information described herein and cause the processes described herein to execute on one or more of the various computers described herein. In one embodiment, the network 306 is configured to transport information according to an Internet Protocol (IP).

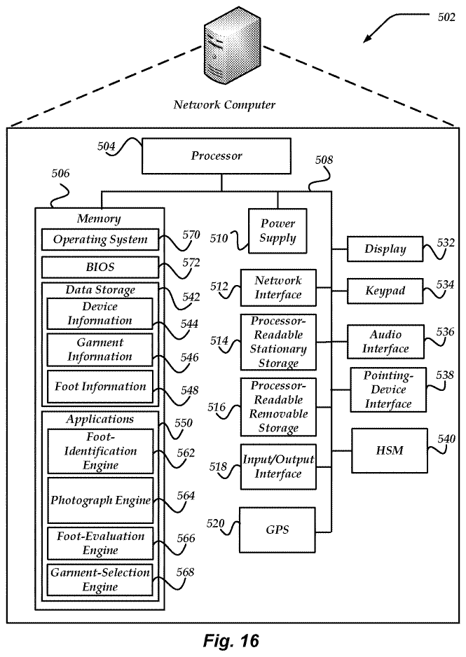

In some embodiments, the system 304 includes one or more of the foot-support selection or generation computer 318, the application server computer 320, or the application server computer 332. One embodiment of the foot-support selection or generation computer 318, the application server computer 320, or the application server computer 332 is described with respect to FIG. 16. Briefly, however, the foot-support selection or generation computer 318, the application server computer 320, or the application server computer 332 is, in some embodiments, configured to host one or more applications and provide one or more services in the network environment. In some embodiments, one or more of the foot-support selection or generation computer 318, the application server computer 320, or the application server computer 332 is configured to implement one or more of the engines of FIG. 1 to implement or execute one or more portions of the processes described herein. In particular, the foot-support selection or generation computer 318 is preferably configured to implement or execute each of the portions of the processes described herein that are not performed by the mobile device. Accordingly, in some embodiments, one or more of the foot-support selection or generation computer 318, the application server computer 320, or the application server computer 332, alone or in combination, facilitate the user obtaining a custom recommendation of a foot-support orthotic by taking only a single photograph of the user's foot or only a single photograph of each of the user's feet with the users ordinary mobile device, such as a mobile smartphone, without placement of a predefined object in the field of view of the camera for scale, such as a piece of paper.

Although FIG. 14 illustrates the foot-support selection or generation computer 318, the application server computer 320, or the application server computer 332 each as a respective single computer, one or more of the foot-support selection or generation computer 318, the application server computer 320, or the application server computer 332 are in some embodiments distributed across one or more distinct network computers. Moreover, in one or more embodiments, one or more of the foot-support selection or generation computer 318, the application server computer 320, or the application server computer 332 are implemented using one or more cloud instances in one or more cloud networks, as shown in FIG. 1.

FIG. 15 schematically shows one embodiment of a client computer 402 that represents, in some embodiments, one or more of the client computers 310-316 in FIG. 14. In some embodiments, the client computer 402 includes a processor 404 in communication with memory 406, such as via bus 408. In some embodiments, the client computer 402 has one or more power supplies 410, network interfaces 412, processor-readable stationary storage devices 414, processor-readable removable storage devices 416, input/output interfaces 418, cameras 420 such as the TrueDepth™ camera system in mobile phones or tablet devices from Apple Inc., video interface 432, touch interfaces 434, hardware security modules (HSM) 436, projectors 438, displays 440, illuminators 442, audio interfaces 444 (e.g., one or more of a display screen, microphone, or speaker), keypads 446, pointing device interfaces 448, global positioning system (GPS) receivers, transmitters, or receivers 450, open-air gesture interfaces 462, sensor interfaces 464 (for example, one or more temperature interfaces, biometric interfaces, accelerometer interfaces, weight scale interfaces, depth measurement devices such as infrared scanners or ultrasonic scanners, or others), or haptic interfaces 466. The client computer 402, in some embodiments, communicates with a base station (not shown) or directly with another computer. In some embodiments, a gyroscope may be employed within the client computer 402 to measure or maintain an orientation of the client computer 402.

In some embodiments, the power supply 410 provides power to the client computer 402 and in some embodiments includes a rechargeable or non-rechargeable battery to provide such power. The power is preferably also or alternatively provided through the power supply 410 by an external power source, such as an alternating-current (AC) adapter or a powered docking cradle that supplements or recharges the battery.

In some embodiments, the network interface 412 includes circuitry for coupling the client computer 402 to one or more networks and is preferably constructed for use with one or more communication protocols or technologies including protocols or technologies that implement one or more portions of the Open Systems Interconnection model (OSI model), such as one or more of global system for mobile communication (GSM), code division multiple access (CDMA), time division multiple access (TDMA), user datagram protocol (UDP), transmission control protocol/Internet protocol (TCP/IP), Short Message Service (SMS), Multimedia Messaging Service (MMS), general packet radio service (GPRS), Wireless Access Point or Wireless Application Protocol (WAP), ultra-wide band (UWB), IEEE 802.16 Worldwide Interoperability for Microwave Access (WiMax), Session Initiation Protocol/Real-time Transport Protocol (SIP/RTP), EDGE, WCDMA, LTE, UMTS, orthogonal frequency-division multiplexing (OFDM), CDMA2000, EV-DO, HSDPA, or other wired or wireless communication protocols. The network interface 412 includes in some embodiments one or more of receivers, transmitters, transceivers, or network interface cards (NIC).

In some embodiments, the audio interface 444 is arranged to produce or receive audio signals such as the sound of a human voice to provide the user with one or more commands to perform one or more of the actions described herein. The audio interface 444 is preferably coupled to or includes one or more of a speaker and microphone (not shown) to in some embodiments enable telecommunication with others or generate an audio acknowledgement for some action. One or more microphones of the audio interface 444 are preferably configured to enable input to or control of client computer 402, such as using voice recognition, detecting touch based on sound, or others.

The display 440 preferably includes a liquid crystal display (LCD), gas plasma, electronic ink, light emitting diode (LED), Organic LED (OLED), or another type of light reflective or light transmissive display that can be used with a computer, such as the projector 438. The display 440 in some embodiments includes the touch interface 434, which is preferably configured to receive input from one or more objects such as a stylus or a digit from a human hand. The touch interface 434 is preferably configured to implement resistive, capacitive, surface acoustic wave (SAW), infrared, radar, or other technologies to sense touch or gestures. In some embodiments, the projector 438 includes a remote handheld projector or an integrated projector that is configured to project an image on a remote wall or any other reflective object such as a remote screen.

The video interface 432 is preferably arranged to capture images, such as one or more still photos, video segments, infrared videos, or others. In some embodiments, the video interface 432 is coupled to one or more digital video cameras, web-cameras, or others. In some embodiments, the video interface 432 has or is coupled to one or more lenses, image sensors, or other electronics. Such image sensors preferably include complementary metal-oxide-semiconductor (CMOS) integrated circuits, charge-coupled devices (CCD), or other circuits for sensing light.

In some embodiments, the keypad 446 includes one or more input devices arranged to receive input from a user, such as one or more push button numeric dials or keyboards. The keypad 446 preferably includes command buttons that are associated with selecting and sending images.

The illuminator 442 preferably provides one or more status indications or otherwise provides light. In some embodiments, the illuminator 442 remains active for specific periods of time or in response to event messages. When the illuminator 442 is active, it preferably backlights buttons of the keypad 446 and in some embodiments stays on while the client computer 402 is powered on. Also, the illuminator 442 preferably backlights those buttons in various patterns when particular actions are performed, such as dialing another client computer. In some embodiments, the illuminator 442 causes light sources positioned within a transparent or translucent case of the client computer 402 to illuminate in response to actions.

The HSM 436 is preferably configured to provide one or more tamper resistant safeguards for generating, storing, or using security or cryptographic information, such as keys, digital certificates, passwords, passphrases, two-factor authentication information, or others. In some embodiments, the HSM 436 is configured to support one or more standard public key infrastructures (PKI) or to generate, manage, or store keys pairs or others. In some embodiments, HSM 436 is implemented as a stand-alone computer or is arranged as a hardware card that is configured to be selectively added to the client computer 402.

The input/output interface 418 is preferably configured to communicate with one or more external peripheral devices or other computers such as other client computers and network computers. The peripheral devices in some embodiments include one or more audio headsets, virtual reality headsets, display screen glasses, remote speaker systems, remote microphone systems, or others. The input/output interface 418 is preferably configured to implement one or more standardized technologies, such as Universal Serial Bus (USB), Infrared, Wi-Fi®, WiMax, Bluetooth®, or others.

The input/output interface 418 preferably includes one or more sensors configured to determine geolocation information (e.g., GPS), monitor electrical power conditions (e.g., one or more voltage sensors, current sensors, frequency sensors, or others), monitor weather (e.g., one or more thermostats, barometers, anemometers, humidity detectors, precipitation scales, or others), or others. Such sensors preferably include one or more hardware sensors that collect or measure data that is external to the client computer 402.

The haptic interface 466 is preferably arranged to provide tactile feedback to a user of the client computer 402. In some embodiments, the haptic interface 466 is configured to vibrate one or more portions of the client computer 402 to notify a user of the client computer 402 of one or more of the events described herein but is preferably configured to not vibrate the client computer 402 while preparing to or during capture of any of the photograph or depth information described herein to prevent moving the client computer 402 during such processes. The sensor interface 464 is preferably configured to provide sensor inputs that facilitate providing the depth information described herein. In some embodiments, instead of the process 92 performing traditional scanning or evaluation processes at block 98, the process 92 proceeds to perform the processes described herein as if the client computer 402 is determined to be capable at block 96 while implementing a sensor connected to the client computer 402 by the sensor interface 464 as if it was native or integral to the camera of the client computer 402. In some embodiments, the one or more sensors are part of the client computer 402. In other embodiments, the one or more sensors are separate and discrete from the client computer 402.

The open air gesture interface 462 is preferably configured to sense physical gestures of a user of the client computer 402, such as through one or more single or stereo cameras, radars, gyroscopic sensors of a computer held or worn by the user, or others. In some embodiments, the camera 420 is configured to track physical eye movements of the user of the client computer 402. In some embodiments, the camera 420 is configured to identify gestures by the user to provide the user inputs described herein such as indicating that the user is prepared for the photograph of the user's foot to be taken by the camera 420 or equipment associated with client computer 402. In some embodiments, the camera 420 is configured to provide depth information with streamed images or photographs taken by the camera 420, such as the TrueDepth™ camera system in mobile phones or tablet devices from Apple Inc. In other embodiments, the camera is configured to provide streamed images or photographs taken by the camera 420, and the computer 402 combine the images or photographs with depth information provided by a sensor connected to the computer 402 by the sensor interface 464, such as an infrared distance or depth scanner or an ultrasonic distance or depth scanner. In some embodiments, one or more applications 468 of the client computer 402 are configured to perform image recognition processes with regard to one or more images of the user's foot captured by the camera 420 to generate one or more values that represent one or more of the measurements described herein. In some embodiments, the one or more images of the foot captured by camera 420 are communicated by the client computer 402 over one or more networks to one or more other computers (for example, one or more network computers 502, the computer 44 or one or more of its engines, the foot-support selection or generation computer 318, the application server computer 320, the application server computer 332, or others) to generate the one or more values that represent characteristics of the user's foot described herein, either based on image recognition processes or user inputs to one or more user interface components of the client computer 402 based on one or more displays of the client computer 402 displaying one or more portions of the one or more images.

The GPS unit 450 is preferably configured to determine the physical coordinates of the client computer 402 on or near the surface of the Earth, which typically outputs a location as latitude, longitude, or elevation values. The GPS unit 450 is preferably configured to implement one or more geo-positioning mechanisms, including triangulation, assisted GPS (AGPS), Enhanced Observed Time Difference (E-OTD), Cell Identifier (CI), Service Area Identifier (SAI), Enhanced Timing Advance (ETA), Base Station Subsystem (BSS), or others, to further determine the physical location of the client computer 402 on or near the surface of the Earth. In one or more embodiments, the client computer 402 is configured to, through one or more other components, provide other information that facilitates determining a physical location of the client computer 402, including a Media Access Control (MAC) address, IP address, or others.

In one or more embodiments, one or more of applications of the client computer 402 (for example, one or more operating systems 470, photograph engines 472, orthotic-selection engines 474, other client applications 476, web browsers 478, or others) are configured to implement geo-location information to select or to enable selecting one or more localization features, such as one or more time zones, languages, currencies, calendar formatting, geographical regions or territories, or others. In some embodiments, localization features are implemented by the client computer 402 or the network computer 502 in one or more portions of file system object meta-data, file system objects, file systems, user-interfaces, reports, internal processes, databases, or others. In some embodiments, the geo-location information is provided by the GPS unit 450. Also, in some embodiments, the geo-location information includes information provided through one or more geo-location protocols over one or more networks, such as wireless network 308, network 306, or others.

In some embodiments, the human-interface components or human-machine interfaces of the client computer 402, such as one or more of the input/output interfaces 418, cameras 420, video interface 432, touch interfaces 434, projectors 438, displays 440, illuminators 442, audio interfaces 444, keypads 446, pointing device interfaces 448, open-air gesture interfaces 462, or haptic interfaces 466, are configured to facilitate presenting the GUI or measurements generated or transmitted by the system 42 of FIG. 1 or providing instructions transmitted by the system 42 of FIG. 1 to personnel for collection of the information transmitted by the client computer to the inventive system in FIG. 1. In some embodiments, one or more human-machine interfaces of the client computer 402 are configured to enable users to manually enter values to modify one or more measurements provided by the system 42. In some embodiments, the human-interface components include peripheral devices that are physically separate from the client computer 402, thereby enabling remote input or output to or from the client computer 402. In some embodiments, one or more of the peripheral interfaces, such as one or more of the input/output interfaces 418, cameras 420, video interface 432, hardware security modules (HSM) 436, audio interfaces 444, GPS units 450, or sensor interfaces 464, are configured to facilitate gathering information from external information or gathering such information from peripheral devices such as distance or depth scanners (e.g., infrared or ultrasonic scanners).

In some embodiments, one or more portions of information described herein as routed through the human-interface components of the client computer 402 such as the display 440 or the keypad 446 is instead be routed through the network interface 412 to one or more human-interface components located remotely. The remote human-interface peripheral components in some embodiments include audio devices, pointing devices, keypads, displays, cameras, projectors, or others, such as the elements of or the devices described herein as coupled to or couplable to the client computer 402. Such peripheral components preferably communicate over a Pico Network such as Bluetooth®, Zigbee™, or others. In some embodiments, such peripheral human-interface components include a wearable computer, which in some embodiments includes one or more remote pico projectors or one or more cameras that remotely communicate with one or more separately located client computers to sense a user's gestures toward one or more portions of one or more image projected by such one or more pico projectors onto one or more reflected surfaces such as a wall, eyewear, a scree, or the user's hand.

The client computer 402 preferably has a web browser application 478 that is configured to receive and to send web pages, web-based messages, graphics, text, multimedia, or others. The browser application 478 preferably is configured to implement one or more programming languages, including a wireless application protocol (WAP) for messages or others. In some embodiments, the browser application 478 is configured to employ Handheld Device Markup Language (HDML), Wireless Markup Language (WML), WMLScript, JavaScript, Standard Generalized Markup Language (SGML), HyperText Markup Language (HTML), extensible Markup Language (XML), HTML5, or others.

In some embodiments, the memory 406 includes one or more of RAM, ROM, or other types of memory. The memory 406 includes one or more computer-readable storage media (devices) for storage of information such as computer-readable instructions, data structures, program modules, or other data. The memory 406 preferably stores one or more BIOSes 480 for controlling low-level operation of the client computer 402. The memory 406 preferably also stores the operating system 470 for controlling the operation of the client computer 402, such as a version of UNIX, LINUX™, Windows®, Windows Phone™, the Symbian® operating system, or others. The operating system 470 preferably includes or interfaces with a Java virtual machine module that enables control of hardware components or operating system operations of the client computer 402 or other computers via Java application programs.

In some embodiments, the memory 406 further includes one or more data storages 492, which are configured to be utilized by the client computer 402 to store, among other things, the applications 468 or other data. In some embodiments, the data storage 492 is configured to store information that describes various capabilities of the client computer 402. The client computer 402 is preferably configured to provide such information to one or more other devices or computers as part of a header during communication, sent upon request, or others. In some embodiments, the data storage 492 is configured to store social networking information such as address books, buddy lists, aliases, user profile information, or others. In some embodiments, the data storage 492 includes program code, data, algorithms, or others for use by a processor, such as processor 404, to execute and perform one or more portions of the actions or processes described herein. In some embodiments, one or more portions of the data storage 492 is located on another component of the client computer 402, including the non-transitory processor-readable removable storage device 416, the processor-readable stationary storage device 414, or one or more components external to the client computer 402.

The data storage 492 preferably includes one or more portions of credential information 494 (e.g., information related to where to provide, bill, or ship the selected or generated foot-support orthotic), foot information 496 (e.g., previously obtained measurements or foot characteristics to enable the user to avoid repeating the process to order a new foot-support orthotic), or orthotic information 498 (e.g., the type of orthotic preferred by the user, such as an insole, orthotic, orthopedic footwear, or others).

The memory 406 in some embodiments includes applications 468 that preferably include computer executable instructions that, when executed by the processor 404, cause the client computer 402 to transmit, receive, or otherwise process instructions and data, including one or more portions of the actions or processes described herein. The applications 468 preferably include one or more photograph engines 472, orthotic-selection engines 474, other client applications 476 such as client portions of one or more of the engines described with respect to one or more of the network computers, web browsers 478, or others that perform one or more portions of the actions or processes described herein. In some embodiments, the photograph engine 472 facilitates obtaining and providing the depth information with the photograph of the user's foot, as described with respect to the customers 46, 50 of FIG. 1, such as with the camera 420 and with or without peripheral devices connected to the client computer 402 by one or more peripheral interfaces such as 418, 446, or 448 or the sensor interface 464. In some embodiments, the photograph engines 472 performs one or more portions of the processes described herein that are typically performed by one or more portions of the computer 44, such as the foot-identification engine 54, the photograph engine 56, or one or more portions thereof. In some embodiments, the orthotic-selection engine 474 facilitates the client computer 402 obtaining orthotic-recommendation reports, alerts, or other notifications generated by the system 42 of FIG. 1 to facilitate the user associated with the client computer 402 approving or ordering the recommended orthotic. In some embodiments, the orthotic-selection engines 474 performs one or more portions of the processes described herein that are typically performed by one or more portions of the computer 44, such as the orthotic-selection engine 54 or one or more portions thereof. In some embodiments, one or more of the applications are executed as or in a web browser.

In one or more of the various embodiments, one or more of the applications 468, such as one or more photograph engines 472, orthotic-selection engines 474, other client applications 476, web browsers 478, or others, are separate and discrete from one or more others of the applications 468. In some of the various embodiments, one or more of the applications 468 include one or more portions of one or more others of the applications 468, such as one or more processes, programming concepts or constructs, or others within one or more of the applications 468. The client computer 402 is preferably configured to exchange communications, such as queries, searches, messages, notification messages, event messages, alerts, performance metrics, log data, API calls, or others, with application servers, network monitoring computers, network computers, other client computers, or others. In some embodiments, the other client applications include one or more calendars, search programs, email client applications, IM applications, SMS applications, Voice Over Internet Protocol (VOIP) applications, contact managers, task managers, transcoders, database programs, word processing programs, security applications, spreadsheet programs, games, search programs, or others.

In one or more embodiments, the processor 404 includes one or more hardware processors or one or more processor cores that are configured to perform one or more portions of the actions or processes described herein. In some embodiments, the client computer 402 includes as the one or more processors 404 one or more embedded logic hardware devices in addition to or instead of one or more CPUs, such as an Application Specific Integrated Circuits (ASICs), Field Programmable Gate Arrays (FPGAs), Programmable Array Logics (PALs), or others. The one or more embedded logic hardware devices are preferably configured to directly execute embedded logic to perform one or more portions of the actions or processes described herein. Also, in one or more embodiments, the client computer 402 includes as the one or more processors 404 one or more hardware microcontrollers instead of or in addition to one or more CPUs. In some embodiments, the one or more microcontrollers are configured to directly execute their own embedded logic to perform one or more portions of the actions or processes described herein and to access its own internal memory and its own external Input and Output Interfaces (e.g., hardware pins or wireless transceivers) to perform actions as a System On a Chip (SOC) or others.

FIG. 16 schematically shows one embodiment of a network computer 502 that represents, in some embodiments, one or more of the network computers 318, 320, 332 in FIG. 14. In some embodiments, the network computer 502 includes a processor 504 in communication with memory 506, such as via bus 508. In some embodiments, the network computer 502 has one or more power supplies 510, network interfaces 512, processor-readable stationary storages 514, processor-readable removable storages 516, input/output interfaces 518, global positioning system (GPS) receivers, transmitters, or receivers 520, displays 532, keypads 534, audio interfaces 536, pointing-device interfaces 538, or HSMs 540. The components of the network computer 502 with names similar to or the same as components of the client computer 402 preferably have similar or the same characteristics, features, or configurations as described herein with respect to such components of the client computer 402, except with the network computer 502 being the point of reference with respect to such characteristics, features, or configurations instead of the client computer 402.

In particular, the human-interface components or human-machine interfaces of the network computer 502, such as one or more of the input/output interfaces 518, displays 532, keypads 534, audio interfaces 536, or pointing-device interfaces 538, are configured to facilitate presenting information to one or more retailers 48 or employees thereof or one or more suppliers 84 employees thereof in the system 42 of FIG. 1 to enable them to follow the instructions for one or more of the processes described herein, manually adjust one of more of the measurements or characteristics identified by one or more of those processes, or selecting or generating the recommended foot-support orthotic.

In some embodiments, the memory 506 includes data storage 542, which stores one or more portions of device information 544 (e.g., the device repository 52 of FIG. 1), orthotic information 546 (e.g., the orthotic-model repository 78 of FIG. 1), or foot information 548 (e.g., the filtered point cloud or evaluated foot characteristics for each of the user's feet from one or more prior evaluations of the user's feet to enable ordering further foot-support orthotics without repeating the photograph or measurement processes). In some embodiments, the memory 506 includes applications 550, such as one or more of a foot-identification engine 562, a photograph engine 564, a foot-evaluation engine 566, a orthotic-selection engine 568, or one or more operating systems 570. In some embodiments, the memory 506 preferably also has one or more BIOSes 572. The applications 550 are preferably configured to cause the network computer 502 to perform one or more portions of the actions or processes described herein when executed by the processor 504.

In some embodiments, one or more of the engines 562, 564, 566, or 568 are configured in the manner described with respect to one or more of the similarly named engines 54, 56, 74, or sub-engines thereof shown in FIG. 1. In some embodiments, one or more of the engines shown in FIG. 16 perform their respective functions by utilizing one or more peripheral devices connected to the network computer 502 by the peripheral or sensor interface 518 or by accessing databases (e.g., the device repository 52 or the orthotic-model repository 78) through the network interface 512. In some embodiments, the network computer 502 collects the photograph with depth information for the user's foot from the client computer 402 or provides the foot information and website code or script to enable the client computer 402 to execute its portions of the one or more processes described herein to the client computer 402 of FIG. 15 through the network interface 512, which in some embodiments, interfaces with the applications 468 in the client computer 402 through the network interface 412 of the client computer 402.

In some embodiments, one or more of the applications 468 or 550 are separate and discrete from one or more of each other. In some embodiments, one or more of the applications 468 or 550 include one or more portions of one or more others of the applications 468 or 550. In some embodiments, one or more of the applications 468 or 550 are implemented as modules or components of another application. Further, in some embodiments, one or more of applications 468 or 550 are implemented as operating system extensions, modules, plugins, or others.

In some embodiments, one or more portions of the applications 468 or 550 are operative in a cloud-based computing environment. In some embodiments, the engines execute within virtual machines or virtual servers that are managed in a cloud-based based computing environment. In some embodiments in this context of cloud-based computing, one or more of the applications 468 or 550 flow from one physical network computer within the cloud-based environment to another depending on performance and scaling considerations automatically managed by the cloud computing environment. In some embodiments, virtual machines or virtual servers dedicated to one or more of the applications 468 or 550 are provisioned and de-commissioned automatically. Also, in some embodiments, one or more of the applications 468 or 550 are located in virtual servers running in a cloud-based computing environment rather than being tied to one or more specific physical network computers. In some embodiments, one or more of the applications 468 or 550 individually or cooperatively perform one or more portions of one or more of the actions or processes described herein, such as one or more actions associated with one or more blocks in one or more of the processes described herein. In some embodiments, one or more of the named engines have sub-engines (not shown) that individually or cooperatively perform one or more portions of the one or more actions. In some embodiments, one or more of the named engines are included as part of another one or more of the other named engines.

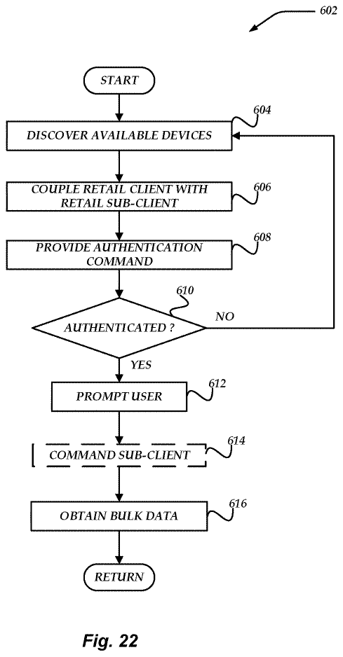

FIG. 22 illustrates a logical flowchart of a process 602 for implementing one or more portions of one or more of the processes described herein in the retail environment according to some embodiments, such as block 100 of the process 92. The process 602 is preferably implemented, performed, or executed by the system 42. One or more elements of the system 42 (e.g., the client device of the retailer 48, the retailer sub-client 49, the foot-identification engine 54, the photograph engine 56, the conversion engine 58, the filter engine 60, the foot-evaluation engine 72, or another component of the computer 44, such as a device of one or more of the suppliers 80) preferably implement, perform, or execute one or more portions of the process 602, as discussed above with respect to FIG. 1. In some embodiments, the process 602 initiates at a start block responsive to a retail employee initiating an instance of an application on the client device 48 or an instance of a companion application on the sub-client device 49.

After the start block, at block 604, the retailer client 48 and the retailer sub-client preferably discover devices that are available for coupling over a network, such as a wireless network like BLE (BLUETOOTH® Low Energy). Some embodiments, the discovery occurs over WI-FI®, and in other embodiments, the devices 48, 49 engage in a handshake with each other with BLE upon selection of one device or the other by the retail employee on the other of such devices, thereby enabling the devices 48, 49 to then automatically discovery each other and couple to each other on a WI-FI® network, such as a WI-FI® network broadcast by one of those devices 48, 49. In the retail environment, however, discovery over WI-FI® is less likely to be available because point-of-sale terminals are on such network, and administrators typically therefore prevent discovery of devices on such networks. Moreover, if the devices 48, 49 couple to each other via WI-FI®, it precludes the devices 48, 49 from communicating over the Internet via such WI-FI® connection. It is therefore preferred that the devices 48, 49 couple to each other via BLE, and the device 48 maintains a connection with the Internet via a separate network, such as a WI-FI® broadcast by a router in the retail environment.

In some embodiments, the sub-client 49 is a device managed by the entity that manages the computer 44 and is provided to the retailer with an instance of an application for executing the sub-client 49 portions of the processes described herein. The client device 48 is preferably a device owned by the retailer, such as a mobile device or desktop computer, and the retailer downloads to the device 48 an application that facilitates controlling and interacting with the sub-client 49 to effectuate the processes described herein.

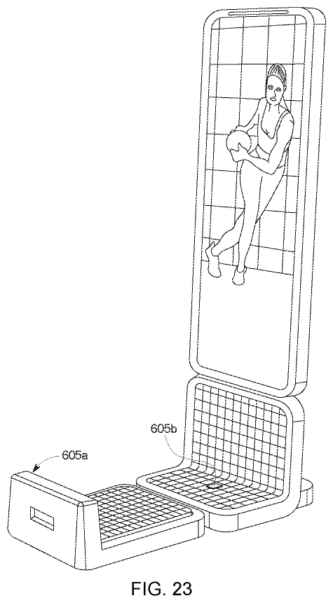

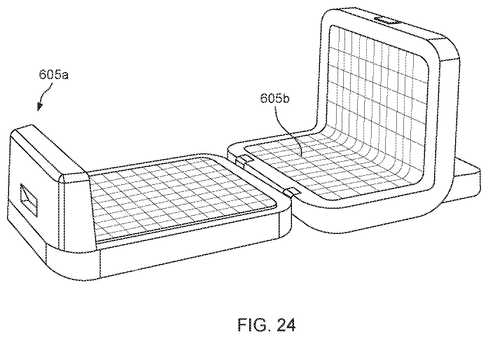

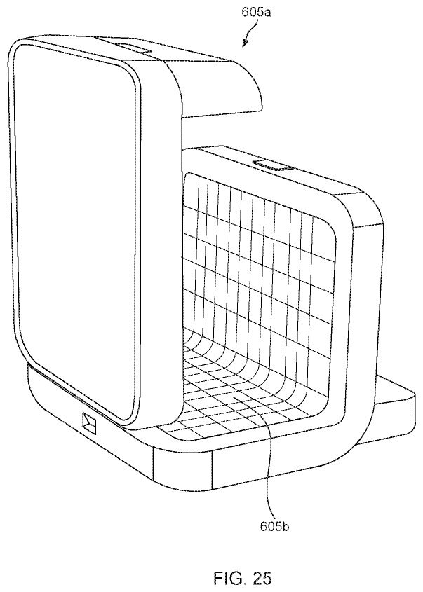

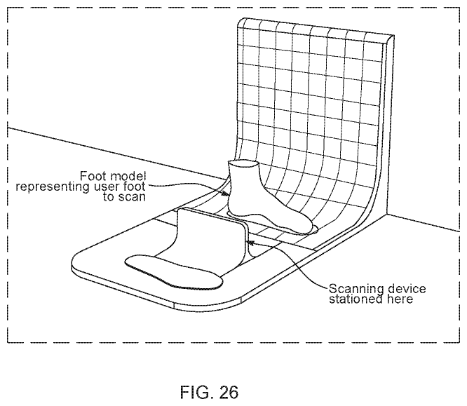

Before or after such discovery, the sub-client device 49 (e.g., an iPhone with a FaceID Camera) is placed on or in a dedicated device holder, such as the holder shown in FIG. 23-25 or the holder shown in FIG. 26. The holder preferably provides power to the device 49 and is plugged into a wall outlet in the retail facility. In some embodiments, the holder optionally includes one or more illuminators such as one or more light emitting diodes (LEDs) that are positioned and oriented to illuminate the customer's foot when placed on a horizontal platform of the holder to facilitate increasing the quality of photographs and information gathered regarding the customer's foot. As shown in FIG. 23, the holder preferably has a housing 605a is sized, dimensioned, and oriented to retain the mobile device 49 with its camera oriented toward a foot placed on a foot platform 605b. In some embodiments, the foot platform 605b or a wall opposite it from the housing 605a optionally has a matrix of dots that facilitate verifying measured characteristics of the customer's foot.

When the application on the sub-client device 49 or the companion application on the client device 48 is launched, such device preferably advertises its availability over BLE.

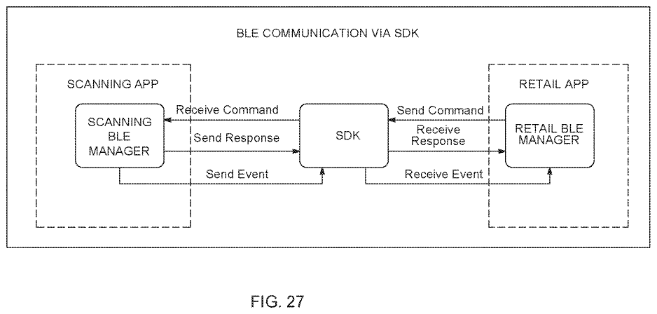

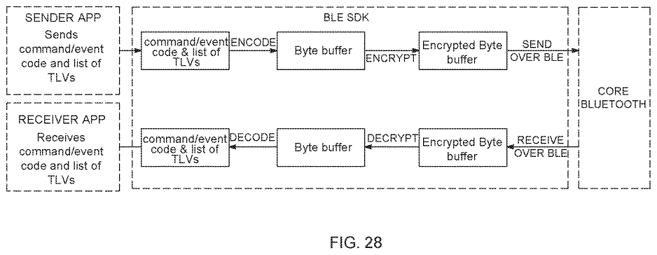

At block 606, the client device 48 and the sub-client device 49 couple to each other, preferably over the wireless network, based on the user (e.g., retail employee) selecting the respective devices 48, 49 on the other of the devices 48, 49. The applications installed in the respective devices 48, 49 preferably provide BLE SDKs (software development kits) as shown in FIGS. 27 and 28 that encapsulate the BLE APIs on the devices 48, 49, such as the iOS BLE APIs to simplify the interface to start discovery over BLE and then provide the devices list to the user. The SDK facilitates the applications providing a command/event as a command code and a list of TLVs (type, length, value) to the SDK, which the SDK encodes and encrypts for sending over BLE to the other device. The SDK of the receiving device receives such data, decrypts and decodes it back to TLVs and makes it available to the application of the receiving device. Accordingly, the SDK encapsulates the BLE interaction complexities and thereby creates a simple interface for communication between the respective applications of the devices 48, 49.

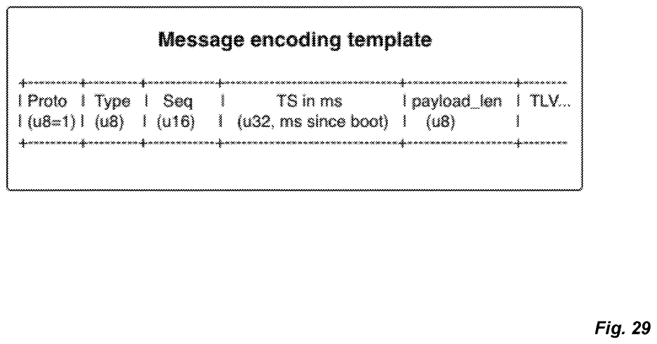

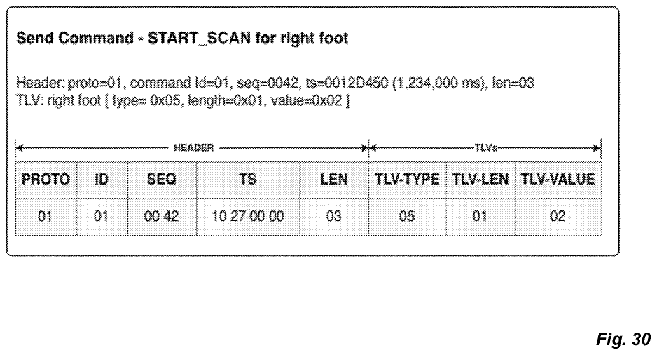

FIGS. 29 and 30 show templates for communication facilitates by the SDK. The SDK encodes the data into a byte buffer. The header includes a protocol version (PROTO), command or event code type or identifier (ID), a sequential value that monotonically increases per send and preferably wraps at 65535 to help detect loss or facilitate reordering data upon receipt out of order (SEQ), a time value of monotonic milliseconds since the app started or the system booted and preferably wraps at a preset value (TS), and a payload length in the number of bytes that follow in the TLV section (LEN). The TLV section includes one or more TLVs. Each TLV includes a type that identifies the TLV type (TLV-TYPE), the length of the TLV or its value length (TLV-LEN), and the value or data of the TLV (TLV-VALUE). In some embodiments, the SDK encrypts the message after encoding it according to the template using the Advanced Encryption Standard Galois Counter Mode (AES-GCM) cipher suite or others. The SDK preferably creates a symmetric key based on a secret key to encrypt the encoded message according to such protocol.

At block 608, the devices 48, 49 exchange authentication commands, such as the client 48 sending an authentication command to the sub-client 49, and the sub-client 49 returning an indication of whether such authentication was successful.

At decision block 610, the devices 48, 49 determine whether the authentication was successful based on the command and success indication exchange. If the authentication succeeds, the devices remain coupled and proceed to block 612; otherwise, the process 602 returns to block 604. In some embodiments, unsuccessful authentication results in the devices 48, 49 blocking each other from transmitting commands to each other, except the authentication request sequence to enable a re-attempt at authenticated connection. Upon successful validation, the application on the sub-client 49 preferably enters a retail experience mode in which it does not audibly communicate prompts to the customer and instead executes its functions and actions responsive to receiving commands over BLE from the client 48. Upon disconnection from the client 48, the application on the sub-client 49 preferably returns to normal mode in which it facilitates direct interaction with the consumer. In some embodiments, a single client 48 couples with and authenticates with multiple sub-clients 49 to facilitate assisting multiple customers at once from a single client 48.

At block 612, the client device 48 provides (e.g., displays) prompts to the user to encourage the retail employee to communicate to the consumer what actions to take to facilitate the scan of the consumer's foot, such as closer, farther, left, right, or others as described regarding other processes herein, such as one or more portions of the process 106, the process 152, or others. The sub-client 49 preferably provides events and information to the client 48 to facilitate determining which prompt to provide to the retail employee, such as whether there was a scan error, whether scan data was captured, a scan result, whether the device 49 is asleep, whether the BLE connection and authentication was successful, when a scan is complete, when a computation of a scan result is complete (e.g., generation of the depth map), whether the device is awake, the device's battery status or a change in such status, a change in the status of a WI-FI® connection, when a scan instruction changes, when a state changes (e.g., idle, active scan, paused scan, processing, result, or error), a countdown to beginning a scan, when an illumination lumen value changes, or others.

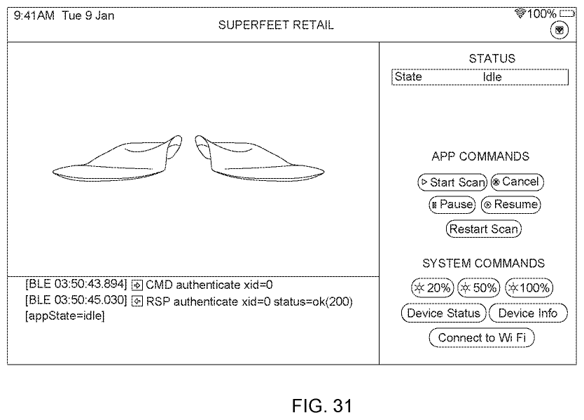

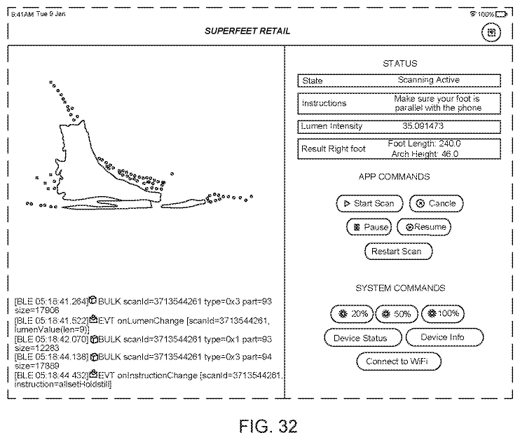

At block 614, the client device 48 optionally provides commands to the sub-client 49. It is optional because the sub-client 49 may automatically execute one or more actions that would correspond to such commands, such as capturing the photograph and depth information pertaining to the foot in response to the foot being detected at an appropriate location, orientation, and position. The client device 48 preferably displays a GUI according to FIG. 31 or 32. As shown in FIG. 31, the GUI enables the retail employee to select one or more commands for controlling the sub-client 49, such as starting a scan of the customer's foot. The GUI preferably displays the status of the sub-client 49 (e.g., idle) and enables or disables various interface components (e.g., changing colors of or graying out buttons) based the status of the sub-client 49 to enable intuitive operation. The GUI facilitates the retail employee providing inputs to the device 48, which are then sent through the SDK to the sub-client 49, preferably as discussed above as a decrypted or encoded TLV over BLE. Commands preferably include begin scan, pause scan, cancel scan, restart scan, increase illumination of the customer's foot, decrease illumination of the customer's foot, or others (see FIGS. 31 and 32). In some embodiments, the commands include overrides of warnings provided by the sub-client 49, such as insufficient light or others.

At block 616, the sub-client 49 provides the client 48 with bulk data that represents characteristics of the customer's foot. In some embodiments, the characteristics include values calculated by the sub-client 49 with respect to the customer's foot, such as length or arch height. Preferably, however, the sub-client 49 provides the photograph with depth information or the point cloud to the client 48. The bandwidth available over a WI-FI® connection is sufficient to allow directly communicating such data from the sub-client 49 to the client 48. In circumstances where the client 48 and the sub-client 49 are coupled to each other over BLE, the bandwidth is significantly more limited. Accordingly, the sub-client 49 provides the bulk data to the client 48 through a chunking and indexing process, preferably with a cyclical redundancy check (CRC), via the SDK and TLV scheme discussed above. In some embodiments, the sub-client 49 provides the initial bulk data to the client 48 with the entire depth map and then only sends incremental changes to the depth data in subsequent messages to preserve bandwidth, such as incremental changes as defined by differences between the previous data packet and the current data packet.

The SDK preferably provides such bulk data from the sub-client 49 to the client 48 at an interval, such as every 2.6 seconds or others, during the transfer over BLE. The SDK preferably encrypts the bulk data and then breaks it into smaller chunks based on a selected Maximum Transfer Unit (MTU), such as 23 bytes for BLE 4.0/4.1 or 251 bytes for BLE 4.2 and 5.0. The chunks are preferably smaller than the MTU by the number of bytes required for the byte buffer to include the header, type, and length. Accordingly, the chunks are preferably smaller than or equal to the MTU minus the sum of the lengths of the header, type, and length as discussed regarding FIGS. 29 and 30. The header is the attached to the chunk with the chunk as the TLV value. The header preferably includes a scan identifier with a value identifying the scan to which the bulk data belongs, a name of the file having the bulk data, an index identifier that identifies the position of the respective chunk in the serial index of chunks being transmitted at each respective interval, a total number of chunks being transmitted, a value identifying the length of the data in the presently transmitted chunk, and a CRC value (e.g., a CRC16 value according to the standards of Comité Consultatif International Téléphonique et Télégraphique (CCITT)). Accordingly, for any given chunk, the combination of the scan identifier, name, and data type in the header is unique. After receipt by the client 48, the SDK validates each chunk using the CRC, continues listening for transmitted chunks until all expected chunks are successfully received, orders them according to the index identification, combines them, and then decrypts the combination to obtain the originally transmitted bulk data.

To reduce the number of chunks and thereby increase the speed of the transfer, the transmitted image is compressed (e.g., reducing size from 1920×1080 pixels to 480×270 and then applying JPEG compression to reduce the image quality 50% and the image size from approximately 3 MB to approximately 12-15 KB). For transmission of the depth map, the depth map is also preferably compressed (e.g., compressed from a resolution of 160×90 to around 16 KB). Both the compressed image and the compressed depth map are thus preferably sent using the SDK's bulk data transfer of block 616. To further reduce the number of chunks and thereby increase the rate of synchronizing information on the client 48 with the sub-client 49, the sub-client 49 preferably sends bulk data for a first frame and then the bulk data transmitted for subsequent frames is limited to the differences between the subsequent frame and the prior frame (i.e., only the first frame is preferably a complete data set).

One or both of the client 48 or the sub-client 49 preferably handles errors with respect to scan errors, connection errors, or others. The scan errors preferably include a detection that the scanning of the customer's foot failed to successfully complete, such as by detecting or determining that there was a cluttered background that prevented an accurate detection of the customer's foot or portions or characteristics thereof, insufficiently complete or accurate depth data, or others. The connection errors preferably include detection that the BLE or WI-FI® connections were interrupted or that multiple such connections were attempted to be made. Other errors preferably include one or more fields missing in a command or other transfer via the SDK, parsing or decoding errors related to transmission or reception via the SDK, an invalid authentication key, scanning errors, missing authentication such as a command or data transfer before authentication completes, or a command conflict such as a command that is unavailable based on a state of the client 48 or sub-client 49. The client 48 preferably also includes a timeout error in the event that it fails to receive within a predefined time a response to a command that it attempted to send to the sub-client 49. The timeout threshold is preferably five seconds, except a ping is preferably three seconds, and connection to WI-FI® is preferably 60 seconds. When a timeout is first detected, the client 48 preferably transmits a ping to the sub-client 49. If the client 48 successfully receives a response from the sub-client 49 before expiration of the ping timeout, the client 48 sends a reset command to the sub-client 49 to instruct it to return to the idle state, thereby synchronizing the states between the applications on the client 48 and the sub-client 49. If the client 48 does not successfully receive a response from the sub-client 49 before expiration of the ping timeout, the process 602 reverts to block 604 to reconnect the devices 48, 49. After authentication following a reconnection, the client 48 sends the reset command to the sub-client 49 to again synchronize the devices 48, 49. In some embodiments, connection errors are handled by the client 48 and the sub-client 49, whereas scan errors are detected and handled by only the sub-client 49, which then notifies the client 48 to instruct the retail employee to work with the customer to initiate a rescan of the customer's foot or feet.

The image of the foot or the depth map or both are preferably displayed on the client 48 (see FIG. 32) and, in some embodiments, while the chunks are received and decoded (see incomplete photo and depth map displayed in FIG. 32).

In some embodiments, the process 602 continues operating until one or more events occur, such as the bulk data being successfully received for a given scan of a foot, both feet of the user, completion of evaluation of one of or both such feet, or others. Next, in some embodiments, control returns to a calling process, such as the process 92, to facilitate presenting a display on the client 48 of the recommended footwear or footwear insert for the retail employee to recommend to the customer and enabling purchase and preferably manufacture of such footwear or footwear insert. In some embodiments, the retail process with the client 48 and sub-client 49 relationship is available to the user on their own devices at home, such as the user's laptop computer operating as the client 48 and the user's iPhone or other mobile device operating as the user's sub-client 49, thereby enabling the user to operate the sub-client 49 without moving it after placement on the wall, as well as confirming, ordering, or purchasing the recommended footwear insert through such client 48. In such at-home experience, the user's client device preferably displays the status of their scan without enabling remote control of the scan while the user controls the scan from the sub-client mobile device.

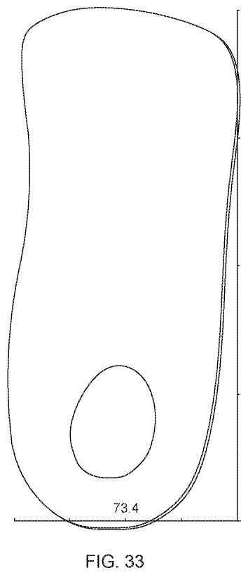

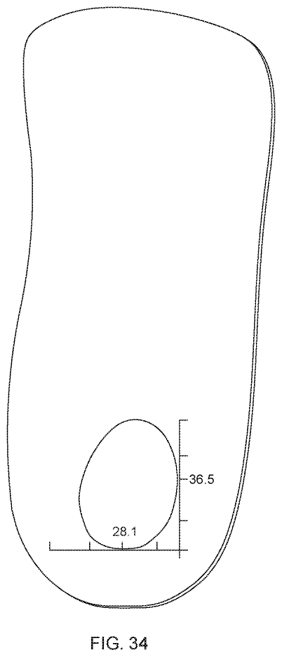

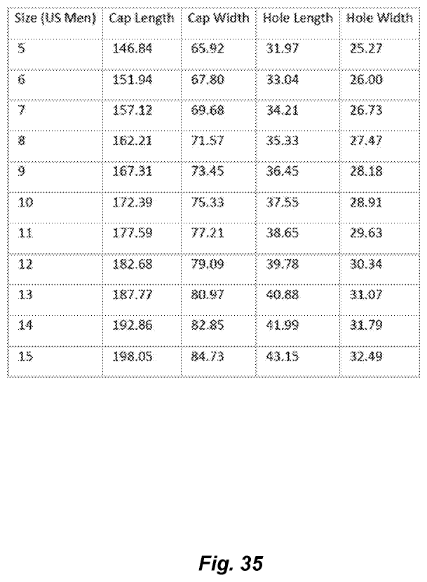

FIGS. 33 and 34 show a preferred model of a footwear insert with axes of several dimensions, such as insert length and insert width (see FIG. 33) and heel opening length and heel opening width (see FIG. 34). The system preferably converts the measured foot length (heel-to-toe) to the nearest insole cap size based in some embodiments on the following lookup table. When the foot length is between two sizes, the system preferably selects the larger of the two sizes.

| Foot Length (mm) | Footwear Insert Size (US men) |
|---|---|
| 228.60 | 5 |
| 237.06 | 6 |
| 245.53 | 7 |
| 254.00 | 8 |
| 262.46 | 9 |
| 270.93 | 10 |
| 279.40 | 11 |
| 287.86 | 12 |
| 296.30 | 13 |
| 304.80 | 14 |
| 313.26 | 15 |

Each footwear insert top cover preferably has length, width, hole length, and hole width dimensions in millimeters as provided in the table below for the size of the insert.

| | Top Cover | Top Cover | Hole | Hole |
|---|---|---|---|---|
| Size (US Men) | Length | Width | Length | Width |
| 5 | 146.84 | 65.92 | 31.97 | 25.27 |
| 6 | 151.94 | 67.80 | 33.04 | 26.00 |
| 7 | 157.12 | 69.68 | 34.21 | 26.73 |
| 8 | 162.21 | 71.57 | 35.33 | 27.47 |
| 9 | 167.31 | 73.45 | 36.45 | 28.18 |
| 10 | 172.39 | 45.33 | 37.55 | 28.91 |
| 11 | 177.59 | 77.21 | 38.65 | 29.63 |
| 12 | 182.68 | 79.09 | 39.78 | 30.34 |
| 13 | 187.77 | 80.97 | 40.88 | 31.07 |
| 14 | 192.86 | 82.85 | 41.99 | 31.79 |
| 15 | 198.05 | 84.73 | 43.15 | 32.49 |

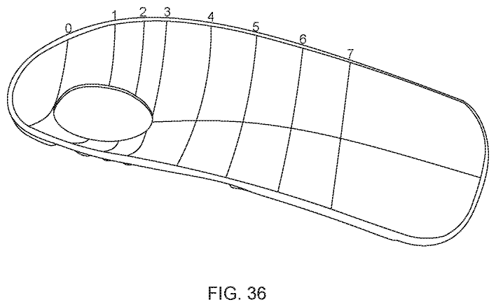

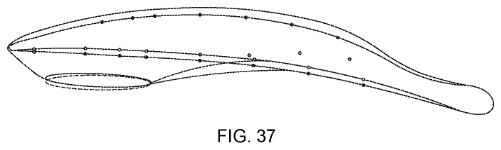

As shown in FIGS. 36 and 37, the footwear insert is preferably customized with respect to its height along its length or width according to the characteristics of the customer's foot as determined by the depth map and processes described herein. The height of the insert is preferably defined according to such customization at the following positions as indicated by a percentage of a length measured from the heel extremity or rearward perimeter of the heel hole to the toes extremity or forward perimeter of the insert. Taking the position numbers shown in FIG. 36, those positions preferably equate to the following percentages. Those longitudinal positions define cross-sections or slices along such length.

| Position Number | Percentage |
|---|---|
| 0 | 0% |
| 1 | 18.5% |
| 2 | 25% |
| 3 | 30% |
| 4 | 40% |
| 5 | 50% |
| 6 | 60% |
| 7 | 70% |

As shown in FIGS. 36 and 37, the footwear insert is preferably customized with respect to its height along its length or width according to the characteristics of the customer's foot as determined by the depth map and processes described herein. The height of the insert is preferably defined according to such customization at the following positions as indicated by a percentage of a length measured from the heel extremity or rearward perimeter of the heel hole to the toes extremity or forward perimeter of the insert. Taking the position numbers shown in FIG. 36, those positions preferably equate to the following percentages. Those longitudinal positions define cross-sections or slices along such length.

As shown by the dots in FIG. 37, the shape of the footwear insole is preferably modified by assigning a height for each slice or cross-section at such slice's or cross-section's respective medial edge, lateral edge, and centerline based on the customer's arch height measured during the scan. In particular, the measured arch height is preferably assigned an arch height for the footwear insert according to the following table, as well as the assignment of a correlated bucket value. If the measured arch height matches a measured arch height in the table, the corresponding insert arch heights and bucket numbers are selected. In some embodiments, the insert arch height is limited to a range of 22 mm-30 mm such that any measured arch height lower than 14 mm will result in selection of an insert arch height of 22 mm, and any measured arch height greater than 42 mm will result in a selected insert arch height of 30 mm. If the measured arch height is 14 mm or greater but less than 18 mm, for example, bucket two is selected. If the measured arch height is 18 mm or greater but less than 22 mm, for example, bucket three is selected.

| Measured Arch | Insert Arch | Bucket |
|---|---|---|
| Height (mm) | Height (mm) | Number |
| <14 | 22 | 1 |
| 14-<18 | 23 | 2 |
| 18-<22 | 24 | 3 |
| 22-26 | 25 | 4 |
| 26-<30 | 26 | 5 |
| 30-<34 | 27 | 6 |
| 34-<38 | 28 | 7 |
| 38-42 | 29 | 8 |
| >42 | 30 | 9 |

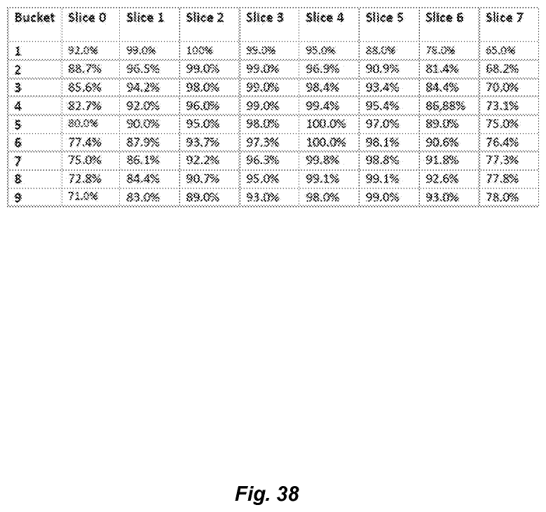

For the medial arch height, the percentages shown in FIG. 38 are preferably multiplied against the selected insert arch height for each slice along the length of the insert based on which bucket is selected to provide the medial arch height at such respective slice. In particular, a selected insert arch height of 23 mm with a selected bucket two provides the following slice values along the medial arch: Slice 0=20.40 mm; Slice 1=22.20 mm; Slice 2=22.77 mm; Slice 3=22.77 mm; Slice 4=22.29 mm; Slice 5=20.91 mm; Slice 6=18.72 mm; and Slice 7=15.69 mm.

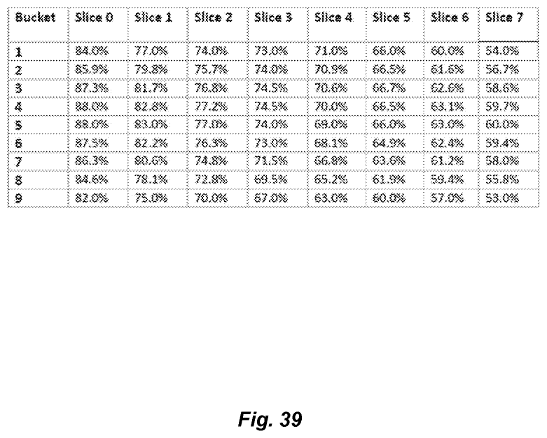

The same approach is preferably taken for the lateral arch height based on the percentages shown in FIG. 39. In particular, taking the selected insert arch height of 23 mm with a selected bucket two provides the following slice values along the lateral arch: Slice 0=17.52 mm; Slice 1=17.72 mm; Slice 2=17.24 mm; Slice 3=16.85 mm; Slice 4=15.80 mm; Slice 5=13.91 mm; Slice 6=11.53 mm; and Slice 7=8.90 mm.

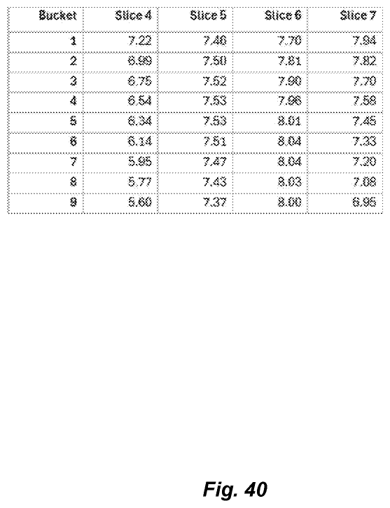

For the centerline height, there is a fixed height for each bucket as shown in millimeters in FIG. 40 (with Slices 0-3 being unadjusted for each arch height because along the centerline Slices 0 and 3 are the perimeter of the heel hole and Slices 1 and 2 are in the heel hole).

Along each of those arch lines (medial, lateral, and centerline), the height of the arch line between the slices is preferably a smooth slope, such as a linear or curved slope.

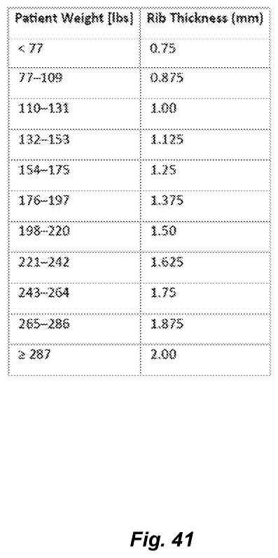

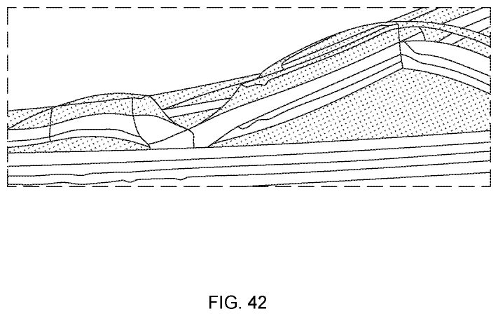

The footwear insert preferably includes a rib height or thickness that corresponds to a patient weight according to the table shown in FIG. 41. Such rib thickness defines a vertical thickness or height of the rib while the ribs preferably retain the same horizontal width for each patient weight. FIG. 42 shows preferred arrangements of ribs superimposed on each other to demonstrate such differences in thickness (dark regions being a shorter rib arrangement with lighter regions being a thicker or higher rib arrangement).

Based on the foregoing, the system or one or more of the clients recommend the customer purchase a footwear insert that corresponds to the values measured, calculated, or selected based on the scan of the customer's one or more feet. Upon selection and purchase by the customer, such footwear insert is preferably selected or created (e.g., additive manufacturing such as 3D printing). Accordingly, the system and processes facilitate providing customized footwear insert that are different for each of the customer's feet.

All the patents, published applications, and non-patent literature referred to in this specification or listed in the information disclosure statement filed herewith are incorporated herein by reference in their entirety, except to the extent that they are inconsistent with the disclosure of this application.

As used herein, the following terms take the meanings explicitly associated herein, unless the context clearly dictates otherwise. The terms “front,” “forward,” “rear,” and “rearward” are defined relative to a direction that a person faces when the person's feet are pointed straight ahead of them such that the toes extended the same direction that the person's torso faces while the heels extended in the opposite direction toward the direction that the person's back faces. The terms “front” and “forward” indicate the direction that the persons toes extend. The terms “rear” and “rearward” indicate the opposite direction to the front or forward direction. The terms “height,” “vertical,” “upper,” “lower,” “above,” “below,” “top,” “bottom,” “topmost,” and “bottom-most” are defined relative to vertical axis of the person's foot when its sole is resting on horizontal ground. The vertical axis is non-parallel to the longitudinal axis and is defined as parallel to the direction of the earth's gravity force on the foot when the foot is on horizontal ground. The term “lateral” is defined relative to the lateral axis of the foot. The lateral axis is non-parallel to the longitudinal and vertical axes. The longitudinal axis extends generally from the heel to the toes.

The term “configured” or “arranged” as used herein means an element being one or more of sized, dimensioned, positioned, oriented, or programmed to achieve or provide the recited function or result. The term “directly coupled” as used herein means that a component contacts (for example, when bolted) or is welded to another component. The term “indirectly coupled” as used herein means that a first component is coupled to a second component by way of one or more intervening components that are directly coupled to the first and second components. A first component that is indirectly coupled to a second component is directly coupled to a third component, which may be directly coupled to the second component or to a fourth component that is directly coupled to the second component. The term “coupled” should therefore be understood to disclose both direct and indirect coupling of components or elements that are described as being coupled to each other.

The term “substantially parallel” refers to parallel or within 5, 10, 15, 20, or 25 degrees of parallel. The term “substantially orthogonal” refers to orthogonal or within 5, 10, 15, 20, or 25 degrees of orthogonal. The term “substantially horizontal” or “substantially horizontally” refers to horizontal or within 5, 10, 15, 20, or 25 degrees of horizontal. The term “substantially vertical” or “substantially vertically” refers to vertical or within 5, 10, 15, 20, or 25 degrees of vertical. The term “approximately” or “substantially” refers to within 1, 5, 10, 15, 20, or 25 percent of the identified value or range.

The phrase “in one embodiment” as used herein does not necessarily refer to the same embodiment or implementation, though it may. Furthermore, the phrase “in another embodiment” as used herein does not necessarily refer to a different embodiment or implementation, although it may. Thus, various embodiments or implementations may be readily combined, without departing from the scope or spirit of the invention.

The term “based on” is not exclusive and allows for being based on additional factors not described, unless the context clearly dictates otherwise. Also, as used herein, the use of “when” and “responsive to” do not imply that associated resultant actions are required to occur immediately or within a particular time period. Instead, they are used herein to indicate actions that preferably occur or be performed in response to one or more conditions being met, unless the context clearly dictates otherwise.

As used herein, the term “foot-identification model” or “foot-identification engine” refers to one or more data structures that encapsulate data, rules, machine learning models, machine learning classifiers, or instructions that are employed to identify an image of a human foot. Such models preferably include various components, such as one or more machine-learning-based classifiers, heuristics, rules, pattern matching algorithms, conditions, or others, that are preferably employed to evaluate pixel data to identify a foot. In some embodiments, such models include, reference, provide one or more inputs or prompts to, or obtain one or more outputs from one or more engines or generative artificial intelligence (“AI”) systems to perform one or more portions of one or more of the actions or processes described herein.

As used herein, the term “engine” refers to logic embodied in hardware or software instructions, such as those written in a programming language including C, C++, Objective-C, COBOL, Java, PHP, Perl, Python, R, Julia, JavaScript, Ruby, VBScript, Microsoft. NET languages such as C#, or others. An engine is preferably compiled into one or more executable programs or written in interpreted programming languages. Software engines are preferably callable from other engines or from themselves. In some embodiments, engines have one or more logical modules that are configured to merge with one or more other engines or applications or to divide into one or more sub-engines. In some embodiments, each of the one or more engines, systems, computers, repositories, or others described herein has one or more respective application programming interfaces (API). In some embodiments, an engine accesses the one or more APIs of one or more engines, systems, computers, repositories, or others described herein to communicate with or provide or obtain data, files, or other information to or from such one or more engines, systems, computers, repositories, or others. In some embodiments, engines include, reference, provide one or more inputs or prompts to, or obtain one or more outputs from one or more other engines discussed herein or generative AI systems to perform one or more portions of one or more of the actions or processes described herein.

As used herein, the term, “configuration information” refers to information that includes one or more rule based policies, pattern matching algorithms, scripts (e.g., computer readable instructions), or others that that are provided from one or more sources, such as one or more configuration files, databases, user inputs, built-in defaults, or others. In some embodiments, configuration information includes or references information stored in one or more other systems or services, such as one or more configuration management repositories, Lightweight Directory Access Protocol (LDAP) servers, name services, public key infrastructure services, or others.

The term “or” is an inclusive grammatical conjunction to indicate that one or more of the connected terms may be employed. For example, the phrase “one or more A, B, or C” or the phrase “one or more As, Bs, or Cs” is employed to discretely disclose each of the following: i) one or more As, ii) one or more Bs, iii) one or more Cs, iv) one or more As and one or more Bs, v) one or more As and one or more Cs, vi) one or more Bs and one or more Cs, and vii) one or more As, one or more Bs, and one or more Cs. The term “based on” as used herein is not exclusive and allows for being based on additional factors not described. The articles “a,” “an,” and “the” include plural references. Plural references are intended to also disclose the singular.

While the preferred embodiment of the invention has been illustrated and described, as noted above, many changes can be made without departing from the spirit and scope of the invention. Each disclosure of a component preferably having a feature or characteristic is intended to also disclose the component as being devoid of that feature or characteristic, unless the principles of the invention clearly dictate otherwise. Accordingly, the scope of the invention is not limited by the disclosure of the preferred embodiment. Instead, the invention should be determined entirely by reference to the claims that follow. It should also be noted that the claim dependencies or combinations of elements recited in the claims do not reflect an intention to forgo claiming other subject matter disclosed herein. Instead, this disclosure is intended to also disclose the subject matter of any combination of any two or more of the claims, such that subsequent claim sets may recite that any one of the dependent claims depends from any other one or more claims, up to and including all other claims in the alternative (such as “The apparatus or method of any one of the preceding or subsequent claims . . . ”). This disclosure is also intended to disclose the subject matter of any one of the dependent claims, as if it were an independent claim, with or without all or a portion of the subject matter of the original independent claim(s) or any other subject matter disclosed herein.

Those of ordinary skill in the art will conceive of other alternate embodiments of the invention upon reviewing this disclosure. Thus, the invention is not to be limited to the above description but is to be determined in scope by the claims that follow.

1. A method comprising:
obtaining an image with depth information for a foot, wherein the image with depth information is captured by a mobile smartphone or a tablet;
based on the image with depth information, evaluating by one or more processors of one or more computers one or more characteristics of the foot;
based on the evaluation, selecting or generating by one or more of the processors of one or more of the computers a foot-support orthotic recommended for the one or more characteristics of the foot,
whereby the evaluation based on the image with depth information captured by the mobile smartphone or tablet facilitates customized selection or generation of the foot-support orthotic with reduced complexity compared to including a non-foot object of a known size in the image as a scaler.

2. The method of claim 1, wherein the depth information includes a depth map provided by the mobile smartphone or the tablet.

3. The method of claim 1, wherein the image with depth information includes only a single still frame photograph with the depth information.

4. The method of claim 3, wherein evaluating the one or more characteristics of the foot is based on only the single still frame photograph with the depth information.

5. The method of claim 1, wherein the image lacks, as the scalar, non-foot objects of size known to the one or more computers evaluating the characteristics of the foot.

6. The method of claim 1, wherein real-time image data from the mobile smartphone or the tablet is evaluated by one or more of the processors of one or more of the computers to determine whether a plurality of parameters are satisfied, and obtaining the image with the depth information for the foot includes one or more of the processors of one or more of the computers automatically causing the mobile smartphone or the tablet to obtain the image with the depth information for the foot based on the determination that the plurality of parameters are satisfied.

7. The method of claim 6, wherein the parameters include that the foot is present in a field of view of a camera of the mobile smartphone or the tablet and that a medial side of the foot faces the camera.

8. The method of claim 1, wherein one or more of the processors of one or more of the computers perform the method in response to the mobile smartphone or the tablet accessing a website.

9. The method of claim 1, wherein one or more of the processors of one or more of the computers perform the method without the mobile smartphone or the tablet downloading an application to enable performance of the method.

10. The method of claim 1, wherein obtaining the image with depth information for the foot includes causing the mobile smartphone or the tablet to display a reference indicator that communicates where the foot should be positioned to facilitate obtaining the image with depth information for the foot.

11. The method of claim 1, wherein the reference indicator is superimposed on a real-time display of a field of view of a camera of the mobile smartphone or the tablet, the position of the reference indicator on the display dynamically varying based on an orientation of the mobile smartphone or the tablet, the dynamic varying including dynamic adjustment of a height of the reference indicator on the display based on an angle of the mobile smartphone or the tablet relative to a floor on which the foot rests.

12. The method of claim 11, wherein the dynamic varying includes dynamic adjustment of the height of the reference indicator on the display based on the angle of the mobile smartphone or the tablet varying relative to the floor while the mobile smartphone or the tablet is in only one of portrait mode or landscape mode.

13. The method of claim 1, further comprising coupling a computer device to the smartphone or tablet, wherein obtaining the image includes controlling the smartphone or tablet with the computer device.

14. The method of claim 1, wherein obtaining the image with depth information includes transmitting a representation of the image with depth information to a remotely located computer device with a BLUETOOTH protocol.

15. The method of claim 14, wherein the BLUETOOTH protocol includes BLUETOOTH LOW ENERGY.

16. The method of claim 14, wherein the transmitting the representation of the image includes dividing an arrangement of bits or bytes into multiple chunks that each have a smaller length than the representation of the image and transmitting the chunks to the computer device with respective index identifiers that enable the computer device to recombine the chunks into an order in which the chunks were arranged by the smartphone or tablet prior to transmission of the chunks.

17. The method of claim 14, wherein transmitting the representation of the image includes transmitting respective representations of multiple images with each representation identifying changes relative to an immediately prior transmitted representation, whereby identification of changes facilitates reducing a number of bits transmitted relative to transmitting an entire representation of an entire image.

18. A method comprising:
obtaining an image with depth information for a foot, wherein the image with depth information is captured by a camera configured to facilitate authorizing access to a device based on biometrics captured by the camera;
based on the image with depth information, evaluating by one or more processors of one or more computers one or more characteristics of the foot;
based on the evaluation, selecting or generating by one or more of the processors of one or more of the computers a foot-support orthotic recommended for the one or more characteristics of the foot.

19. The method of claim 18, wherein the camera includes a TrueDepth camera system.

20. The method of claim 18, wherein the image with depth information includes only a single still frame photograph with the depth information.

21. The method of claim 20, wherein evaluating the one or more characteristics of the foot is based on only the single still frame photograph with the depth information.

22. The method of claim 18, wherein the image lacks, as the scalar, non-foot objects of size known to the one or more computers evaluating the characteristics of the foot.

23. The method of claim 18, wherein real-time image data from the camera is evaluated by one or more of the processors of one or more of the computers to determine whether a plurality of parameters are satisfied, and obtaining the image with the depth information for the foot includes one or more of the processors of one or more of the computers automatically causing the camera to obtain the image with the depth information for the foot based on the determination that the plurality of parameters are satisfied.

24. The method of claim 18, wherein the parameters include that the foot is present in a field of view of the camera and that a medial side of the foot faces the camera.

25. The method of claim 18, wherein one or more of the processors of one or more of the computers perform the method in response to the mobile smartphone or the tablet accessing a website.

26. The method of claim 18, wherein a scaler is included in the image and one or more characteristics of the foot are verified based on the scaler in the image.
